SEQUENCE LISTING (5 sequences)

SEQ ID NO: 1 - DNA, 1968 bp, Homo sapiens
atggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtag

SEQ ID NO: 2 - protein, 655 aa, Homo sapiens
  SIGNAL (1)..(41)
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 3 - protein, 631 aa, Homo sapiens [sequence omitted]

SEQ ID NO: 4 - DNA, 2009 bp, Homo sapiens
agctttctggggcaggccaggcctgaccttggctttggggcagggagggggctaaggtgaggcaggtggcgccagcaggtgcacacccaatgcccatgagcccagacactggacgctgaacctcgcggacagttaagaacccaggggcctctgcgcctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagcaggctcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtc

SEQ ID NO: 5 - protein, 330 aa, Homo sapiens [sequence omitted]

(19) United States
(12) Patent Application Publication
(10) Pub. No.: US 2004/0043022 A1
(43) Pub. Date: Mar. 4, 2004

(54) Treating t-cell mediated diseases by modulating dr6 activity

(76) Inventors: Josef Georg Heuer, Indianapolis, IN (US); Jinqi Liu, Plainsboro, NJ (US); Songqing Na, Carmel, IN (US); Ho Yeong Song, Carmel, IN (US); Derek Di Yang, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/257,907

(22) PCT Filed: Apr. 30, 2001
(86) PCT No.: PCT/US01/11735

(51) Int. Cl. A61K 39/395; A61K 31/00

(52) U.S. Cl. 424/143.1; 514/1

(57) ABSTRACT

Novel methods are provided for the treatment or prevention of T cell mediated conditions in a mammal that comprise administering to said mammal a therapeutically effective amount of a pharmaceutical composition comprising at least one DR6 agonist or DR6 antagonist.

### FIELD OF THE INVENTION

The present invention relates generally to recombinant DNA technology as applied to the field of human medicine. In particular, the invention relates to new methods of treating or preventing at least one symptom associated with immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, and/or eczema in a mammal.

### BACKGROUND OF THE INVENTION

Tumor necrosis factor (TNF) is a key mediator involved in immune regulation and inflammation. TNF family members mediate their biological functions through structurally related, but functionally distinct receptors that belong to the TNF receptor (TNFR) family. The interactions between TNF family ligands and their receptors are involved in modulating a number of signaling pathways in the immune system such as proliferation, differentiation, apoptosis and cell survival (Wallach, D., et al., Annu. Rev. Immunol., 17:331-367 (1999)). The factors that are involved in regulating T cell proliferation and differentiation have yet to be completely elucidated. However, at least two signals have been demonstrated to be required for T cell activation. The first signal is provided by T cell receptor (TCR) interaction with peptide/MHC complexes and the second signal is provided by co-receptor interaction with accessory molecules on the antigen presenting cells (Lenschow, D., et al., Annu. Rev. Immunol., 14:233-258 (1996)). Ligation of CD28 by B7-1 (CD80) and/or B7-2 (CD86) molecules have been well characterized as predominant co-stimulatory interactions which increase IL-2 production and T cell proliferation. In vivo studies have also shown that CD28/CD86-mediated co-stimulation may be critical for Th2 development (Khattri, R., et al., J. of Immunology, 162:5784-5791. (1999)). Recent data has also suggested that other molecules act either in a synergistic pathway with CD28, or in a CD28-independent manner (Watts, T., et al., Current Opinion in Immunology, 11:286-293 (1999)). In addition, in vitro studies have suggested that an efficient T cell response requires the combined use of several of ligand-receptor pairs including several TNF/TNFR family members such as CD40, CD27, CD30, 4-1BB, and OX-40. These molecules have been shown to deliver a co-stimulatory signal for T cell proliferation, cytokine production, or Th1/Th2 differentiation when engaged by their corresponding ligands or specific antibodies (Abs) (Gravestein, L. A. et al., Seminars in Immunology, 10:423-434 (1998)). Data generated using CD40- and CD40L-deficient mice have indicated that the interactions between these molecules are essential for an optimal T cell response (Noelle, R., et al., Immunity, 4: 415-419 (1996)). TNFRII has been reported to stimulate proliferation of human T cells (Tartaglia, L., et al., 151:4637-4641 (1993)). CD30, when expressed on an activated T cell, serves as a positive regulator to enhance the proliferation of T cells in response to CD3 cross-linking (Gilfillan, M. C., et al., 160:2180-2187 (1998)). 4-1BB has been reported to promote Th1 cell responses by enhancing IL-2 and IFN-γ and suppressing IL-4 production in vitro (Saoulli, K., et al., J. Exp. Med., 187:1849-1862 (1998)). 4-1BB cross-linking with an anti-4-1BB monoclonal antibody (mAb) preferentially activates CD8⁺ T cells both in vitro and in vivo. Cytokine analysis of in vitro activated CD4⁺ and CD8⁺ T cells revealed that anti-4-1BB co-stimulation significantly enhance IFN-γ production by CD8⁺ T cells (Watts, T. H., et al., Current Opinion in Immunology, 11:286-293 (1999)). OX-40 and CD28 signals are highly synergistic for CD4⁺ T cell proliferation and survival. Reportedly, OX-40 signals act as the potent co-stimulator for a developing primary CD4 response and for late phase continued proliferation and expansion of effector cells and OX-40-L-deficient mice engendered suppression of T cell recall response after being primed with protein antigens and allo-antigens (Chen, A. I., et al., Immunity, 11:689-698 (1999); Kopf, M., et al., 11:699-708 (1999)). Moreover, it has also been demonstrated that OX-40/OX-40L interactions are involved in mediating Th2 development in vivo (Ohshima, Y., et al., Blood, 92:3338-3345 (1998)). This finding suggested that other regulatory pathways may exist that promote Th2 development in the absence of CD28 and consistent with the studies using CD28-deficient BALB/c mice to indicate that CD28 is not an absolute requirement for Th2 development (Lenschow, D. J., et al., Immunity, 5:285-293 (1996)). In short, observations from these studies strongly suggest that TNF/TNFR family members act as important accessory molecules for promoting the most effective T cell immune responses when combined with TCR signals. However, while many TNF/TNFR family members appear important for positive regulation of T cell immune responses, negative regulatory molecules among the family have yet to be elucidated.

DR6 was identified as another death domain containing receptor (Pan, G., et al., FEBS, 431:351-356 (1998)). The extracellular cysteine-rich domain of DR6 has about 40% homology with osteoprotegerin (OPG) and TNFR2. However, the physiological functions of DR6 remain unknown. The present invention concerns novel methods of treating various mammalian diseases using agonists and antagonists of DR6.

### BRIEF SUMMARY OF THE INVENTION

Applicants disclose herein that in mice DR6 mRNA is expressed in resting T cells and down regulated in activated T cells. Although DR6 deficient mice appear to develop normally, DR6−/−CD4⁺ T cells hyper-proliferate in response to TCR-mediated stimulation and protein antigen challenge. Furthermore, as compared to activated wild-type CD4⁺ T cells, activated DR6−/−CD4⁺ T cells exhibit upregulated CD25 expression, enhanced proliferation in response to exogenous IL-2 stimulation, upregulated CD28 expression, and downregulated CTLA-4 expression. Activated DR6−/−CD4⁺ T cells also demonstrate enhanced Th2 cytokine production associated with increased levels of the transcription factor NF-ATc in nuclei as compared to activated wild-type CD4⁺ T cells. Applicants are the first to identify DR6 as the first TNFR family member to act as a negative mediator of alloantigenic response in mammals. Accordingly, the present invention relates generally to methods for treating or preventing conditions and/or diseases involving loss or deterioration of immune competence, on one hand, or immune overactivity on the other. More particularly, the present invention concerns novel methods of modulating T cell proliferation, differentiation, and/or activation that comprise the administeration of a biologically effective amount of a DR6 agonist or a DR6 antagonist.

It is an object of the present invention to provide methods for treating or preventing immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, eczema, and/or at least one condition or symptom related thereto, in a mammal that comprise administering to said mammal a therapeutically effective amount of a pharmaceutical composition comprising at least one DR6 agonist or DR6 antagonist.

One embodiment of the present invention provides a method of treating or preventing immunodeficiency, cancer, bacterial or viral infection, complications of bacterial or viral infection, and/or at least one condition or symptom related thereto, in a mammal that comprises administering to said mammal a therapeutically effective amount of a pharmaceutical composition comprising at least one DR6 antagonist.

Another embodiment of the present invention provides a method of treating or preventing autoimmunity, GVHD, T-cell mediated cytotoxicity, inflammatory bowel diseases, T-cell mediated inflammatory diseases, apoptosis, asthma, allergy, atopy, eczema, and/or at least one condition or symptom related thereto, in a mammal that comprises administering a therapeutically effective amount of a pharmaceutical composition comprising a DR6 agonist

It is also an object of this invention to provide methods for enhancing cell mediated immunity in a mammal that comprise administering a therapeutically effective amount of a pharmaceutical composition comprising at least one DR6 antagonist

It is also an object of this invention to provide methods for inhibiting cell mediated immunity in a mammal that comprise administering a therapeutically effective amount of a pharmaceutical composition comprising at least one DR6 agonist.

### BRIEF DESCRIPTION OF THE DRAWINGS

FIG. 1 depicts the gene targeting method used to generate DR6-deficient mice. The pGT-N29-tk vector, the targeting vector pKO-DR6, the genomic region at the DR6 locus, and the predicted structure of the mutated DR6 gene are illustrated. The restriction fragments used to construct the targeting vector are indicated (1.2-kb StuI-XbaI fragment and 3.5-kb BamHI-EcoRV fragment). The coding regions of the exons are presented as striped. Restriction enzyme sites (B, BamHI; N, NotI; Ns, NsiI; P, PstI; R, EcoRI; RV, EcoRV; Stu, StuI; Xba, XbaI) and the probe A for Southern blot analysis are indicated.

FIG. 2 (A) shows the Southern Blot analysis of mouse genomic DNA indicating germline transmission of the DR6 disruption as evidenced by the presence of all three expected genotypes (the upper band (4.7kb) corresponds to the wild-type allele, and the lower band (3.1kb) corresponds to the mutant allele) and (B) shows the Northern Blot analysis of poly A+ mRNA (0.5 μg per lane) from kidneys of 5-week-old mice confirming the absence of DR6 transcripts in DR6 deficient mice. mRNA was hybridized to a 524 b.p. DR6 cDNA probe derived from exon 4 of full-length DR6 cDNA.

FIG. 3 depicts the enhanced proliferation of peripheral DR6 deficient T cells in response to antiTCR/anti-CD28 or anti-TCR stimulation. For graph (A) purified T cells from spleen were cultured for 48 h in a 96 well plate coated with different concentrations of anti-CD3 and anti-CD28. [³H]thymidine was added to the culture for the last 12 h of incubation. The amount of [³H] incorporation was examined and used to determine the proliferation of peripheral T cells in response to cross-linking TCR and CD28. For graph (B) IL-2 secretion from the purified T cells was examined by ELISA after 24 h stimulation with 1 μg/ml anti-CD3/anti-CD28. For graph (C) purified T cells from spleen were cultured in the presence of different concentrations of anti-CD3 for 48 h. The data represents five independent experiments. DR6 deficient T cells were labeled as DR6 KO for this and all following figures.

FIG. 4 illustrates the enhanced proliferation of DR6 deficient CD4⁺ T cells. For graph (A) purified sub-populations of T cells were cultured for 48 h in a 96 well plate coated with 1 μg/ml anti-CD3 and anti-CD28. Proliferation was determined after 48 hrs by [³H] thymidine incorporation. For graph (B) soluble DR6-Fc and IgG1 were used to treat wild type T cells to block the interaction between DR6 ligand and DR6 in the presence of 0.2 μg/ml anti-CD3. The cells were treated for 48 hrs and pulsed with [³H]-thymidine. For graph (C) soluble DR6-Fc and IgG1 were used to treat wild type CD4⁺ T cells to block the interaction between DR6 ligand and DR6 in the presence of 0.2 μg/ml anti-CD3. The above data represents five independent experiments. For graph (D) real-time PCR was invoked to determine the expression level of DR6 in CD4⁺ T cells before and after stimulation with 1 μg/ml anti-CD3/anti-CD28 at different timepoints. For graph (E) the percentage of cell undergoing apoptosis was determined by staining with Annexin V and measurement by flow cytometry.

FIG. 5 graphically illustrates the enhanced in vivo CD4⁺ T cell priming, in vitro T cell recall responses and cytokine productions that result in the absence of DR6. For graph (A) CD4⁺ wild type mice and DR6 deficient mice were immunized with KLH in the hind footpads. Nine days after immunization, draining lymph nodes were extracted and subjected to an in vitro challenge with various concentrations of KLH in the presence of antigen presenting cells. After culturing for 4d, [³H]-thymidine uptake was measured. For graph (B) wild type and DR6 deficient mice were immunized with the protein antigen HEL, and CD4⁺ T cell recall responses were measured as for (B). For graph (C) the supernatants from in vitro T cell recall responses of wild type and DR6 deficient mice were collected and IL-4, IL-5, IL-10, and IFN-γ levels were measured by ELISA.

FIG. 6 (A) shows an immunoblot analysis of the protein levels of NF-ATc and NF-kB p50 in the nuclear extracts made from lymph node cells before and after stimulation with anti-TCR/anti-CD28 (1 μg/ml) for 72 h. The gel mobility shift assay shown in (B) suggests that the increased amount of nuclear NF-ATc from activated DR6 deficient T cells was functional. The middle bands were non-specific binding since they were not competed by cold homologous oligonucleotide.

FIG. 7 is a graph depicting (A) morbidity and mortality and (B) body weight changes of BDF1 recipients after adoptive transfer of allogeneic wild type, DR6 deficient (DR6 KO T cells) and T cell-depleted splenocytes in a murine parent-into-F1 model of acute GVHD. BDF1 mice were irradiated with a dose sufficient to temporarily ablate the immune system.

FIG. 8 illustrates the enhanced destruction of double-positive thymocytes by transplantation of DR6 deficient T cells in allogeneic recipients. On day 7 after transplantation, cell numbers in recipient thymus were counted (A) and the size of thymus from each transplantation group was compared (B).

FIG. 9 shows engraftment of purified T cells from splenocytes of wild type and DR6 deficient mice when injected i.v. into irradiated (5Gy) allogenic BDF1 recipient mice. BDF1 mice that received donor T cell-depleted splenocytes served as a negative control. On day seven after transplantation, cells from spleens of recipient mice were examined for percentage of engraftment of donor CD4, CD8 T cells (A) and destruction of host B cells was also measured by two-color immunofluorescent staining and flow cytometry (B).

FIG. 10 shows DR6 deficient T cells display an increased expansion in mixed lymphocyte stimulation reactions (A) and increased cytotoxicity against host antigens compared with wild-type T cells (B).

FIG. 11 depicts serum TNF-α and IFN-γ levels of recipient mice at 7 days post-transplant. While recipients having wild type donors showed significant increases in compared to TCD recipient mice, recipients having DR6 deficient donors had significantly higher serum cytokine levels than recipients of T cells from wild-type mice.

### DETAILED DESCRIPTION OF THE INVENTION

### General Definitions

The term “amino acid” is used herein in its broadest sense, and includes naturally occurring amino acids as well as non-naturally occurring amino acids, including amino acid variants and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid variants and derivatives; naturally occurring non-proteogenic amino acids such as norleucine, β-alanine, ornithine, etc.; and chemically synthesized compounds having properties known in the art to be characteristic of amino acids.

The incorporation of non-natural amino acids, including synthetic non-native amino acids, substituted amino acids, or one or more D-amino acids into the polypeptides used herein may be advantageous in a number of different ways. D-amino acid-containing polypeptides exhibit increased stability in vitro or in vivo compared to L-amino acid-containing counterparts. Thus, the construction of polypeptides incorporating D-amino acids can be particularly useful when greater stability is desired or required in vivo. More specifically, D-peptides are resistant to endogenous peptidases and proteases, thereby providing improved bioavailability of the molecule, and prolonged lifetimes in vivo when such properties are desirable. When it is desirable to allow the polypeptide to remain active for only a short period of time, the use of L-amino acids therein will permit endogenous peptidases and/or proteases to digest the molecule, thereby limiting the cell's exposure to the molecule. Additionally, D-peptides cannot be processed efficienty for major histocompatibility complex class II-restricted presentation to T helper cells, and are therefore less likely to induce humoral immune responses in the whole organism.

In addition to using D-amino acids, those of ordinary skill in the art are aware that modifications in the amino acid sequence of a polypeptide can result in functional sDR6 polypeptides or DR6 antibodies that display equivalent or superior functional characteristics when compared to the original polypeptide or antibody. Thus, the methods of the present invention contemplate the use of modified sDR6 polypeptides and/or DR6 antibodies. Contemplated modifications in the sDR6 polypeptides and/or DR6 antibodies useful in the present invention may include one or more amino acid insertions, deletions, substitutions, truncations, fusions, shuffling of subunit sequences, and the like, either from natural mutations or human manipulation, provided that the sequences produced by such modifications have substantially the same (or improved or reduced, as may be desirable) activity(ies) and/or utilities as the sDR6 polypeptides or DR6 antibodies utilized in the methods disclosed herein.

The term “antagonist” in reference to a polypeptide is used in the broadest sense and includes any molecule that partially or fully blocks, inhibits, decreases, or neutralizes a biological activity of a polypeptide. Furthermore, the term antagonist is intended to include any molecule that partially or fully blocks, inhibits, decreases the expression of a polypeptide encoding polynucleotide or a polypeptide. As defined here antagonists include nucleotide sequences, such as anti-sense and ribozyme molecules, and gene or regulatory sequence replacement constructs that can be used to inhibit expression of a messenger RNA transcript coding for a polypeptide. As used herein, the term “antagonist” in reference to DR6 is also meant to include soluble forms of the DR6 polypeptide that are able to compete for the binding of DR6 agonistic ligands and agonistic anti-DR6 antibodies to DR6. In a similar manner, the term “agonist” in reference to a polypeptide is used in the broadest sense and includes any molecule that induces or increases the expression of a polypeptide encoding polynucleotide or induces or increases the stability and/or biological activity of a polypeptide. An agonist may include for example, small molecules, naturally occurring ligand agonists, polypeptide ligand agonists, and antibodies specific for an epitope of the polypeptide.

The term “tumor necrosis factor receptor” and “TNFR” when used herein encompass native TNFR sequences, including those found in a variety of mammals, including humans. The TNFR may be isolated from a variety of sources, such as from human tissue types or from another source, or prepared by recombinant or synthetic methods. Collectively, members of the TNFR family of receptors mediate a variety of biological effects of TNF ligands, including inhibition of bone resorption (by virtue of inhibiting osteoclast differentiation), inflammatory processes, apoptosis, and protection against infection and induction of shock.

The term “DR6” refers to a nucleic acid, gene, cDNA (e.g., SEQ ID NO:1) and fragments thereof as well as any polypeptide encoded thereby (e.g., SEQ ID NOS:2 and 3) as reported by Pan et al., (1998) and which appears in the GenBank database as accession no. AF068868. DR6 is also known in the art as TR7 which was described in EP0869179 A1. A member of the TNF receptor superfamily, full-length DR6 contains 655 amino acids. The term “DR6” without further limitation is meant to include both the native or full-length polypeptide (SEQ ID NO:2) as well as the mature form of DR6.

The phrase “DR6 antibody” refers to an antibody as defined herein that recognizes and binds at least one epitope of a DR6 polypeptide.

The term “sDR6” refers to a soluble form of DR6 that, when expressed, is secreted (i.e., is not membrane bound) and contains at least a functional extracellular domain. A sDR6 polypeptide may additionally contain other non-extracellular domain sequences provided that the polypeptide remains non-membrane bound. As a skilled artisan recognizes that a polynucleotide encoding DR6, or an appropriate fragment thereof, is a starting material for preparing a sDR6 polypeptide.

Furthermore, DR6 encoding polynucleotides can be isolated from nature or can be produced by recombinant or synthetic means as a source of obtaining the extracellular domain or other polypeptide segments comprising a particular sDR6. sDR6, as referred to herein, includes, but is not limited to, deglycosylated, unglycosylated, and modified glycosylated forms of sDR6, as well as, forms having conservative substitutions, additions, or deletions of the amino acid sequence, or portions thereof, such that the sDR6 retains a capability of binding its natural ligand.

In addition to the ability to bind a natural ligand, the extracellular domain of a sDR6 can be identified by the presence of at least one homologous “cysteine-rich domain” in the DR6 protein. As used herein, the term “cysteine-rich domain” refers to a sDR6 domain having an amino acid sequence of at least about 20, preferably at least about 30, and more preferably at least about 35-40 amino acid residues which contain at least about 2, 3, 4, 5, or 6 cysteine residues. Larger cysteine rich domains of about 45 to 50 or 60 amino acid residues will have up to 7, 8, 9, or 10 cysteine residues. The TNFR cysteine rich domains of SEQ ID NOS:2 or 3 and related sequences occur from about amino acids 39 to about 76, 77 to about 118, 119 to about 162, and 163 to about 201 of SEQ ID NO:2. Preferred sDR6 molecules have an amino acid sequence sufficiently homologous to a cysteine rich domain amino acid sequence of SEQ ID NO:2 from about amino acid residue 42 to about 350, more narrowly from about 42 to about 211 through 214. As used herein, the term “sufficiently homologous” refers to a first amino acid or nucleotide sequence which contains a sufficient or minimum number of identical or or related amino acid substitutions (for related amino acids see Table 1 for conservative substitutions and discussion of groups, infra.) or nucleotides to a second amino acid or nucleotide sequence such that the first and second amino acid or nucleotide sequences have a common structural domain and/or common functionality. Preferably, a sufficiently homologous polypeptide comprises a extracellular domain having at least about 85% homology, more preferably at least about 90% homology, more preferably at least about 95% homology, more preferably at least about 96% homology, more preferably at least about 97% homology, more preferably at least about 98% homology, more preferably at least about 99% homology, and most preferably 100% homology to the ECD of DR6 as described herein. Preferably, a sufficiently homologous polynucleotide comprises a polynucleotide encoding an extracellular domain having at least about 85% homology, more preferably at least about 90% homology, more preferably at least about 95% homology, more preferably at least about 96% homology, more preferably at least about 97% homology, more preferably at least about 98% homology, more preferably at least about 99% homology, and most preferably 100% amino acid homology to the ECD of DR6 as described herein. The term “sDR6” is also meant to encompass a DR6 ECD fused to pro- or prepro-sequences, processing of which will result in the production of a “sDR6”. The natural leader sequence of human DR6 linked to the DR6 ECD would be considered as being included within the definition of sDR6. sDR6 molecules may additionally contain other DR6 sequences provided that the molecule still retains a functional extracellular domain and is soluble. Similarly, molecules having additional sequences not naturally part of DR6 may be fused to a DR6 ECD and still fall under the definition of sDR6.

As indicated in SEQ ID NO:2, the native signal peptide of DR6 is encoded thereby from about amino acid position 1 (methionine-1) to about amino acid position 41 (alanine-41). Alternatively, the signal peptide is encoded thereby from about amino acid position 25 (methionine-25) to about amino acid position 41 (alanine-41). It should be noted, however, that the C-terminal boundary of either putative signal peptide may vary, but likely by no more than about 5 amino acids, preferably, by no more than about 4 amino acids, more preferably, by no more than about 3 amino acids, more preferably, by no more than about 2 amino acids, and more preferably, by no more than about 1 amino acid.

The transmembrane domain of DR6, has been putatively identified as occurring from about amino acid positions 339 through 351 to about amino acid positions 360 through 370 of SEQ ID NO:2. This region serves to anchor DR6 to the membrane. Preferred sDR6 polypeptides would be deleted of critical amino acids within the TMD, more preferred sDR6 polypeptides would be deleted of substantially all amino acids comprising the TMD domain, and most preferred sDR6 polypeptides would be deleted of all amino acids comprising the DR6 TMD domain.

The cytoplasmic domain of DR6 occurs from about amino acid positions 361 through 371 to about amino acid 655 of SEQ ID NO:2. sDR6 molecules optionally may comprise the cytoplasmic domain or fragments thereto.

The term “sDR6” is also intended to encompass chimeric protein molecules not found in nature comprising a translational fusion, or in some cases, an enzymatic fusion in which two or more different proteins or fragments thereof are covalently linked on a single polypeptide chain. However, the resulting polypeptide chain must comprise at least one functional fragment of the extracellular domain of DR6. Additional sequences that may be added to the DR6 extracellular domain include, but are not limited to, encoded signal peptide (SP) sequences, encoded “non-functional” fragments of the transmembrane domain (TMD), and encoded cytoplasmic domain (CD) sequences such that the resulting molecule containing the added homologous sequences is functionally a soluble receptor as defined herein. The fusion molecules are a subclass of chimeric polypeptide fusions of sDR6 molecules which additionally contain a portion of an immunoglobulin sequence (herein referred to sDR6-Ig). The chimeric sDR6-Ig fusions may also comprise forms in monomeric, homo- or heteromultimeric, and particularly homo- or heterodimeric, or homo- or heterotetrameric forms; optionally, the chimeras may be in dimeric forms or homodimeric heavy chain forms. Tetrameric forms containing a four chain structural unit are the natural forms in which IgG, IgD, and IgE occur. A four-chain structure may also be repeated. Different chimeric forms containing a native immunoglobulin are known in the art (WO 98/25967). The mature human protein of Example 12 is an exemplary of a “sDR6-Ig.” As used herein, the term “sDR6-Ig” designates antibody-like molecules that combine at least one natural ligand binding region of the extracellular binding domain of a sDR6 with the effector functions of immunoglobulin constant domain. Structurally, sDR6-Ig molecules comprise an amino acid sequence with the natural binding capacity of a sDR6 fused to an immunoglobulin constant domain sequence. The extracellular domain part of the molecule is typically a contiguous amino acid sequence of sDR6 comprising at least a functional extracellular domain containing the binding site to a natural ligand. The immunoglobulin constant domain sequence may be obtained from any immunoglobulin, such as IgG-1, IgG-2, IgG-3 or IgG-4 subtypes, IgA (including IgG-1 and IgA-2), IgE, IgD or IgM. Preferred fusions contain the sDR6 fused to the amino terminus of the Ig region. Fusions of sDR6 to the C-terminus of a Ig region are also contemplated. sDR6 molecules can also comprise additional amino acid residues, such as affinity tags that aid in the purification or identification of the molecule or provide sites of attachment to a natural ligand.

As used herein, the term “functional” in reference to a sDR6 or the phrase “functional extracellular domain” of DR6 indicates that the fragment of DR6 retains the capacity to bind at least one natural DR6 ligand and therefore is able to compete for ligand binding with endogenously expressed, membrane bound DR6 polypeptides. Such functionality may be evidenced by in vitro or in vivo assays of DR6 activity conducted with use or administration of the putatively functional sDR6 as compared to controlled the same assay conducted without the use or without administering the putatively functional sDR6. The term “selectively binding” refers to the ability of a DR6 agonist or DR6 antagonist to selectively bind DR6 or a natural ligand of DR6, whether such binding is demonstrated in vitro or in vivo binding assays.

The term “inhibit” or “inhibiting” includes the generally accepted meaning, which includes prohibiting, preventing, restraining, lessening, slowing, stopping, or reversing, including, but not limited to, in reference to the progression or severity of a disease or condition or biological consequence.

In the present disclosure, “isolated” refers to material removed from its original environment (e.g., the natural environment if it is naturally occurring), and thus is altered “by the hand of man” from its natural state. For example, the term “isolated” in reference to a polypeptide refers to a polypeptide that has been identified and separated and/or recovered from at least one contaminant from which it has been produced. Contaminants may include cellular components, such as enzymes, hormones, and other proteinaceous or non-proteinaceous solutes. Ordinarily, however, isolated polypeptides will be prepared by at least one purification step.

The term “isolated” in reference to a nucleic acid compound refers to any specific RNA or DNA molecule, however constructed or synthesized or isolated, which is locationally distinct from its natural location. For example, an isolated polynucleotide could be part of a vector or a composition of matter, or could be contained within a cell, and still be “isolated” because that vector, composition of matter, or particular cell is not the original environment of the polynucleotide.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. Ordinarily, an isolated antibody is prepared by at least one purification step. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain. An “isolated antibody” is also intended to mean an antibody that is substantially puriefied from other antibodies having different antigenic specificities (e.g., an isolated antibody that specifically binds DR6 substantially purified antibodies that specifically bind epitopes other than those of DR6). An isolated antibody that specifically binds DR6 epitopes may bind DR6 homologous molecules from other species.

“Soluble receptor” as used herein refers to a receptor molecule that is not bound to a cell membrane. Soluble receptors are most commonly ligand-binding receptors polypeptides that lack a functional transmembrane domain and often lack the cytoplasmic domain. Receptor polypeptides are termed soluble when they are unable to provide a membrane anchoring function. Generally, a soluble receptor results when essential amino acids anchoring the molecule to the cell membrane have been deleted. The remaining sequences of the transmembrane domain (TMD) which lack the ability to hold the protein to the membrane are referred to herein in as “non-functional fragments” of the TMD.

The phrase “substantially pure” or “substantially purified” may be used interchangeably with the term “isolated” in reference to a macromolecule that is separated from other cellular and non-cellular molecules, including other proteins, lipids, carbohydrates or other materials with which it is naturally associated when produced recombinantly or synthesized without any general purifying steps. A “substantially pure” preparation or a substantially purified preparation would be about at least 85% pure; preferably about at least 95% pure. A “substantially pure” or “isolated” protein as described herein could be prepared by a variety of techniques well known to the skilled artisan. In preferred embodiments, a polypeptide will be purified (1) to greater than 95% by weight of polypeptide to the weight of total protein as determined by the Lowry method, and most preferably to more than 99% by weight of polypeptide to the weight of total protein, (2) to a degree sufficient to obtain at least 15 residues of N-terminal or internal amino acid sequence by use of a spinning cup sequenator, or (3) to apparent homogeneity by SDS-PAGE under reducing or nonreducing conditions using Coomassie Blue, or preferably, silver stain, such that the major band constitutes at least 95%, and, more preferably 99%, of stained protein observed on the gel.

General Recombinant DNA and Protein Terms

“Conservative substitution” or “conservative amino acid substitution” refers to a replacement of one or more amino acid residue(s) in a protein or peptide. Conservative substitutions of interest are shown in Table 1 along with preferred substitutions. If such substitutions maintain or improve the desired function, then more substantial changes, then listed as exemplary substitutions in Table 1, or as further described below in reference to amino acid classes, are introduced and the products screened. A substitution a particular position can constitute can be 1, 2, 3, 4, 5, 10, 15, 20 amino acids.

TABLE 1
Conservative Substitutions

| Original | Exemplary | Preferred |
|---|---|---|
| Residue | Substitutions | Substitutions |
| Ala | val, leu, ile | val |
| Arg | lys, gln, asn | lys |
| Asn | gln | gln |
| Asp | glu | glu |
| Cys | ser | ser |
| Gln | asn | asn |
| Glu | asp | asp |
| Gly | pro, ala | ala |
| His | asn, gln, lys, arg | arg |
| Ile | leu, val, met, ala | leu |
| Leu | norleucine, ile, | ile |
| Lys | arg, gln, asn | arg |
| Met | leu, phe, ile | leu |
| Phe | leu, val, ile, ala, | leu |
| Pro | ala | ala |
| Ser | thr | thr |
| Thr | ser | ser |
| Trp | tyr, phe | tyr |
| Tyr | trp, phe, thr, ser | phe |
| val | ile, leu, met, phe, | leu |

Naturally occurring residues are divided into groups based on common side-chain properties:

(1) hydrophobic: cys, ser, thr;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

An “effective amount” of a DR6 agonist or DR6 antagonist is the minimal amount of the compound that must be delivered to result in a measurable modulation of at least one DR6 associated activity.

“Fragment thereof” refers to a fragment, piece, or sub-region of a nucleic acid or protein molecule whose sequence is disclosed herein, such that the fragment comprises 5, 10, 15, 20 or more amino acids, or 15, 30, 45, 60 or more nucleotides that are contiguous in the parent protein or nucleic acid compound. When referring to a nucleic acid compound, “fragment thereof” refers to 15, 30, 45, 60 or more contiguous nucleotides, derived from the parent nucleic acid, and also, owing to the genetic code, to the complementary sequence. For example if the fragment entails the sequence 5′-AGCTAG-3′, then “fragment thereof” would also include the complementary sequence, 3′-TCGATC-5′.

“Functional fragment” or “functionally equivalent fragment,” as used herein, refers to a region, or fragment of a full length protein, or sequence of amino acids that, for example, contains an a functional ligand binding site, or any other conserved ligand binding motif, relating thereto. As used herein functional fragments of a sDR6 polypeptide are capable of competiting for the binding of a natural ligand to a natural or recombinantly expressed DR6 polypeptide.

The term “fusion protein” denotes a hybrid protein molecule not found in nature comprising a translational fusion or enzymatic fusion in which two or more different protein segments not naturally found in a contiguous sequence are covalently linked together, generally on a single peptide chain.

Also as used herein the term “abnormal apoptosis” refers to excessive and/or improper apoptosis. Typically abnormal apoptosis is observed in cells and tissues that have undergone physical, chemical or biological insult. Such insults include, but are not limited to, physical injury, viral infection, bacterial infection, ischemia, irradiation, chemotherapy, and the like.

The term “half-life” as used herein refers to the time required for approximately half of the molecules making up a population of said molecules to be cleaved in vitro. More specifically, “plasma half-life” refers to the time required for approximately half of the molecules making up a population of said molecules to be removed from circulation or be, otherwise, rendered inactive in vivo.

The term “homolog” or “homologous” describes the relationship between different nucleic acid compounds or amino acid sequences in which said sequences or molecules are related by partial identity or similarity at one or more blocks or regions within said molecules or sequences.

“Host cell” refers to any eukaryotic or prokaryotic cell that is suitable for propagating and/or expressing a cloned gene contained on a vector that is introduced into said host cell by, for example, transformation or transfection, or the like.

The term “hybridization” as used herein refers to a process in which a single-stranded nucleic acid compound joins with a complementary strand through nucleotide base pairing. The degree of hybridization depends upon, for example, the degree of homology, the stringency of hybridization, and the length of hybridizing strands. “Selective hybridization” refers to hybridization under conditions of high stringency.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. However, cleavage sites for a secreted protein may be determined experimentally by amino-terminal sequencing of the one or more species of mature proteins found within a purified preparation of the protein.

The term “naturally occurring” is used to designate that the object it is applied to, e.g., naturally occurring ligand, can be isolated from a source in nature.

A “nucleic acid probe” or “probe” as used herein is a labeled nucleic acid compound that hybridizes with another nucleic acid compound. “Nucleic acid probe” means a single stranded nucleic acid sequence that will combine with a complementary or partially complementary single stranded target nucleic acid sequence to form a double-stranded molecule. A nucleic acid probe may be an oligonucleotide or a nucleotide polymer. A probe will usually contain a detectable moiety which may be attached to the end(s) of the probe or be internal to the sequence of the probe.

The term “plasmid” refers to an extrachromosomal genetic element. The plasmids disclosed herein are commercially available, publicly available on an unrestricted basis, or can be constructed from readily available plasmids in accordance with published procedures.

A “primer” is a nucleic acid fragment which functions as an initiating substrate for enzymatic or synthetic elongation of, for example, a nucleic acid compound.

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

“Recombinant DNA cloning vector” as used herein refers to any autonomously replicating agent, including, but not limited to, plasmids and phages, comprising a DNA molecule into which one or more additional DNA segments can be or have been incorporated.

The term “recombinant DNA expression vector” or “expression vector” as used herein refers to any recombinant DNA cloning vector (such as a plasmid or phage), in which a promoter and other regulatory elements are present, thereby enabling transcription of an inserted DNA, which may encode a polypeptide.

The term “stringency” refers to hybridization conditions. High stringency conditions disfavor non-homologous base-pairing. Low stringency conditions have the opposite effect. Stringency may be altered, for example, by temperature and salt concentration.

“Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration, for example 1 M NaCl.

“High stringency” conditions comprise, for example, a temperature of about 42° C. or less, a formamide concentration of less than about 20%, and a low salt (SSC) concentration; or, alternatively, a temperature of about 65° C., or less, and a low salt (SSPE) concentration. For example, high stringency conditions comprise hybridization in 0.5M NaHPO₄, 7% sodium dodecyl sulfate (SDS), 1 mM EDTA at 65° C. (Ausubel, F. M. et al., Current Protocols in Molecular Biology, Vol. I, 1989; Green Inc., New York, at 2.10.3).

“SSC” comprises a hybridization and wash solution. A stock 20×SSC solution contains 3M sodium chloride, 0.3M sodium citrate, pH 7.0.

“SSPE” comprises a hybridization and wash solution. A 1×SSPE solution contains 180 mM NaCl, 9 mM Na₂HPO₄, 0.9 mM NaH₂PO₄ and 1 mM EDTA, pH 7.4.

The term “vector” as used herein refers to a nucleic acid compound used for introducing exogenous or endogenous DNA into host cells. A vector comprises a nucleotide sequence which may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes are carried out according to the manufacturer's recommendation.

Immunoglobulin Terminology

In the present disclosure the term “antibody” is intended to refer to a monoclonal antibody per se, or an immunologically effective fragment thereof. Antibodies may or may not be glycosylated, though glycosylated antibodies are preferred. Antibodies are properly cross-linked via disulfide bonds, as is well known. It has been shown that the antigen-binding function of an antibody can be performed by fragments of a full-length antibody. Therefore, the phrases “antibody fragment”, “antibody portion”, “antigen binding portion” or the phrase “fragment thereof” in reference to an antibody includes fragments of an antibody that retain the ability to specifically bind to an antigen. Examples of binding fragments encompassed within these terms include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein, chain in which the VL and VH regions pair to form monovalent molecules (known as single chain Fv(scFv): see e.g., Bird et al. (1988) Science 242:423-426; and Huston et al. (1988) Proc. Natl. Acad. Sci. USA 85:5879-5883). Also included within the definition “antibody” for example, are single chain forms, generally designated Fᵥ regions, of antibodies. It is intended also that such single chain antibodies are encompassed within the term “antigen-binding portion” of an antibody or “antibody fragment”. Other forms of single chain antibodies, such as diabodies are similarly encompassed with the definition of the term “antibody”. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Hollinger P., et al. (1993) Proc. Natl. Acad. Sci. USA 90:6444-6448; Plijak, R. J., et al. (1994) Structure 2:1121-1123). Still further, an antibody or antigen-binding portion thereof may be part of a larger immunoadhesion molecule, formed by covalent or non-covalent association of the antibody or antigen binding portion with one or more other proteins or peptides. Examples of such immunoadhesion molecules include use of the streptavidin core region to make a tetrameric scFv molecule (Kipriyanov, S. M., et al (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al (1994) Mol. Immunol. 31:1047-1058). Antibody fragments, such as Fav and F(ab′)2 fragments, can be prepared from whole antibodies using conventional techniques, such as papain or pepsin digestion, respectively, of whole antibodies. Moreover, antibodies, antibody fragments, and immunoadhesion molecules can be obtained using standard recombinant DNA techniques as known in the art or as described herein. Unless stated otherwise, as long as the immunoglobulin protein demonstrates the ability to specifically bind its intended target, in this case, DR6 polypeptides, it is included within the term “antibody” as used herein. In particular, human and humanized antibodies are included within the meaning of the term “antibody”.

CDR1, CDR2, or CDR3 of the heavy chain variable region alternatively are referred to hereinafter as H1, H2, and H3 respectively, and the CDR1, CDR2, and CDR3 of the light chain variable region are referred to hereinafter as L1, L2, and L3, respectively, of an antibody.

The term “human antibody” includes antibodies having variable and constant regions corresponding to human germline immunoglobulin sequences as described by Kabat et al. (1991). The human antibodies of the invention may include amino acid residues not encoded by human germline immunoglobulin sequences (e.g., mutations introduced by random or site-specific mutagenesis in vitro or by somatic mutation in vivo), for example in the CDRs and, in particular, CDR3. Any human antibody can also be substituted at one or more positions with an amino acid, e.g., a biological property enhancing amino acid residue, which is not encoded by the human germline immunoglobulin sequence. In preferred embodiments, these replacements are within the CDR regions as described in detail below.

Human antibodies have at least three advantages over non-human and chimeric antibodies for use in human therapy:

1) because the effector portion of the antibody is human, it may interact better with the other parts of the human immune system (e.g., destroy the target cells more efficiently by complement-dependent cytotoxicity (CDC) or antibody-dependent cellular cytotoxicity (ADCC);

2) The human immune system should not recognize the human antibody as foreign, and therefore the antibody response against such an injected antibody should be less than against a totally foreign non-human antibody or a partially foreign chimeric antibody;

3) injected non-human antibodies have been reported to have a half-life in the human circulation much shorter than the half-life of human antibodies. Injected human antibodies will have a half-life essentially identical to naturally occurring human antibodies, allowing smaller and less frequent doses to be given.

The term “activity” in reference to DR6 or a DR6 polypeptide or the phrases “DR6 associated activity” or “DR6 biological activity” is intended to mean, for example, inhibition of T cell proliferation in response to exogenous IL-2 stimulation, inhibition of T-cell mediated cytotoxicity, enhancement of CTLA-4 expression in activated CD4⁺ T cells, or inhibition of CD28 expression and/or CD25 expression in activated CD4⁺ T cells. Accordingly, DR6 activity or DR6 associated activity can be assessed by one or more of the in vitro or in vivo assays disclosed herein or otherwise known in the art. The term “activity” in reference to a DR6 agonist or DR6 antagonist includes, but is not limited to, the ability to agonize or antagonize at least one DR6 biological activity or DR6 associated biological activity.

The term “neutralizing”, “antagonizing”, “antagonistic” in reference to an anti-DR6 antibody or the phrase “antibody that antagonizes DR6 activity” is intended to refer to an antibody or antibody fragment whose binding to DR6 results in inhibition of a DR6 biological activity or a DR6 associated biological activity. Similarly, the term “agonistic” or “agonist” in reference to an anti-DR6 antibody, small molecule, or naturally occurring DR6 ligand is intended to refer to one that enhances or stimulates DR6 associated biological activity. The effects of a putative DR6 agonist or a DR6 antagonist on DR6 activity or a DR6 associated activity can be assessed by measuring the effect of a putative DR6 agonist or a putative DR6 antagonist on one or more in vitro or in vivo indicators of DR6 activity.

The term “epitope tagged” where used herein refers to a chimeric polypeptide comprising a sDR6 polypeptide fused to a “epitope tag”. The epitope tag has enough residues to provide an epitope against which an antibody may be made, or which can be identified by some other agent, yet is short enough such that it does not interfere with the activity of the sDR6 polypeptide. The eptitope tag preferably is also fairly unique so that the antibody does not substantially cross-react with other epitopes. Suitable tag polypeptides generally have at least six amino acid residues and usually between about 8 to about 50 amino acid residues, or more preferably, between about 10 to about 20 residues.

The term “HIS tag” where used herein refers to the sDR6 sequence, or portion thereof, fused to a highly rich histidine polypeptide sequence. The HIS tag has enough histidine residues to provide a unique purification means to select for the properties of the repeated histidine residues, yet is short enough such that it does not interfere with the activity of the extracellular domain sequence of sDR6. Suitable tag polypeptides generally have at least six amino acid residues and usually between about 4 to about 20 amino acid residues (preferably, between about 4 to about 10 residues, and most preferably 6, such as HHHHHH). Several codons encoding a hemagglutinin fragment (i.e., an “HA” tag to facilitate purification) or HIS tag (see, e.g., Ausubel et al., ed., Current Protocols in Molecular Biology, John Wiley and Sons, NY (1987-1999)) followed by a termination codon and polyadenylation. The HA tag corresponds to an epitope derived from the influenza hemagglutinin polypeptide (Wilson et al., Cell 37:767-778 (1984)). The fusion of the HA tag to the target polypeptide allows easy detection and recovery of the recombinant polypeptide with an antibody that recognizes the HA epitope.

Pharmaceutical Terms

The term “administer” or “administering” means to introduce by any means a therapeutic agent into the body of a mammal in order to prevent or treat a disease or condition.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Administration “in combination with” one or more further therapeutic agents includes simultaneous (concurrent) and consecutive administration in any order.

A “therapeutically-effective amount” is the minimal amount of a compound or agent that is necessary to impart therapeutic benefit to a mammal. By administering graduated levels of a DR6 agonist or DR6 antagonist to a mammal in need thereof, a clinician skilled in the art can determine the therapeutically effective amount of the DR6 agonist or DR6 antagonist required for administration in order to treat or prevent an immunodeficiency, cancer, bacterial or viral infection, complications of bacterial or viral infection, complication of infection, autoimmunity, GVHD, T-cell mediated cytotoxicity, apoptosis, asthma, allergy, atopy, eczema, and/or at least one symptom thereof. Such determinations are routine in the art and within the skill of an ordinarily skilled clinician.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS™.

“Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “mammal” as used herein refers to any mammal, including humans, domestic and farm animals, and zoo, sports or pet animals, such as cattle (e.g. cows), horses, dogs, sheep, pigs, rabbits, goats, cats, and non-domesticated animals like mice and rats. In a preferred embodiment of the present invention, the mammal being treated or administered to is a human or mouse.

“Symptom associated with” in reference to a disease or condition such as immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, and/or eczema may include one or more of the following: chills, profuse sweating, itching, fever, weakness, hypotension, leukopenia, intravascular coagulation, shock, respiratory distress, organ failure, prostration, ruffled fur, diarrhea, eye exudate, and death, alone or in combination. This list is not meant to be exclusive, but may be supplemented with symptoms or combinations of symptoms that a person of ordinary skill would recognize as associated with immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, and/or eczema. Symptoms associated with immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, and/or eczema that are treatable DR6 agonists or DR6 antagonists are within the scope of definition. A symptom associated with immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, and/or eczema may also be associated with another condition.

As used herein, “Th1-mediated” in reference to a disease, disorder, or condition is one that has been associated with increased cytokine production from Th1 cells, including IFN-γ, IL-2, GM-CSF, TNF-•, and IL-3. Specific examples include multiple sclerosis, insulin-dependent diabetes mellitus, Crohn's disease, uveitis, chronic rheumatism, and systemic lupus erythematosus.

As used herein, “Th2 mediated” in reference to a disease, disorder, or condition is one that has not been associated with increased cytokine production from Th1 cells. Specific examples include scleroderma, multiple myositis, vasculitis syndrome, mixed connective tissue disease, Sjögren's syndrome, hyperthyroidism, Hashimoto's disease, myasthenia gravis, Guillain-Barré syndrome, autoimmune hepatopathy, ulcerative colitis, autoimmune nephropathy, autoimmune hematopathy, idiopathic interstitial pneumonia, hypersensitivity pneumonitis, autoimmune dermatosis, autoimmune cardiopathy, autoimmune infertility, and Behcet's disease.

All references to a disease or condition are contemplated to encompass other diseases, conditions, and/or symptoms associated with the referenced disease or condition by the medical community. For instance, the phrase “autoimmune disease(s)” is used herein to refer to a large group of illnesses, some with ill-defined causes, thought to be associated with abnormalities in immunoregulation. Therefore, the term as used herein is intended to include, but is not limited to, diseases such as rheumatoid arthritis, lupus, graft versus host disease, host versus graft disease, insulin-dependent diabetes, autoimmune encephlomyelitis, autoimmune hepatitis, Crohn's disease, and multiple sclerosis. Additionally, the term “allergy” is meant to encompass allergic disease(s) including, but not limited to, chronic bronchitis, atopic dermatitis, pollinosis (allergic rhinitis), allergic angiitis, allergic conjunctivitis, allergic gastroenteritis, allergic hepatopathy, allergic cystitis, and allergic purpura.

“Pharmacologically effective amount” or “physiologically effective amount” of a DR6 agonist or DR6 antagonist is the amount of the compound needed to provide a desired level of the compound in the mammal to be treated to give an anticipated physiological response when it is administered, such as intravenously, subcutaneously, intraperitoneally, orally, or through inhalation. The precise amount of the compound required to be pharmacologically effective will depend upon numerous factors, e.g., such as the specific binding activity of the compound, the delivery device employed, physical characteristics of the compound, purpose for the administration, in addition to patient specific considerations. The amount of a compound that must be administered to be pharmacologically effective can be be determined by one skilled in the art without undue experimentation.

A “small molecule” is defined herein to have a molecular weight below about 500 daltons.

The terms “treating”, “treatment” and “therapy” as used herein refer to curative therapy, prophylactic therapy, and preventative therapy. An example of “preventative therapy” is the prevention or lessening of a targeted disease or related condition thereto. Those in need of treatment include those already with the disease or condition as well as those prone to have the disease or condition to be prevented. The terms “treating”, “treatment”, and “therapy” as used herein also describe the management and care of a mammal for the purpose of combating a disease, or related condition, and includes the administration of sDR6 to alleviate the symptoms or complications of said disease, condition.

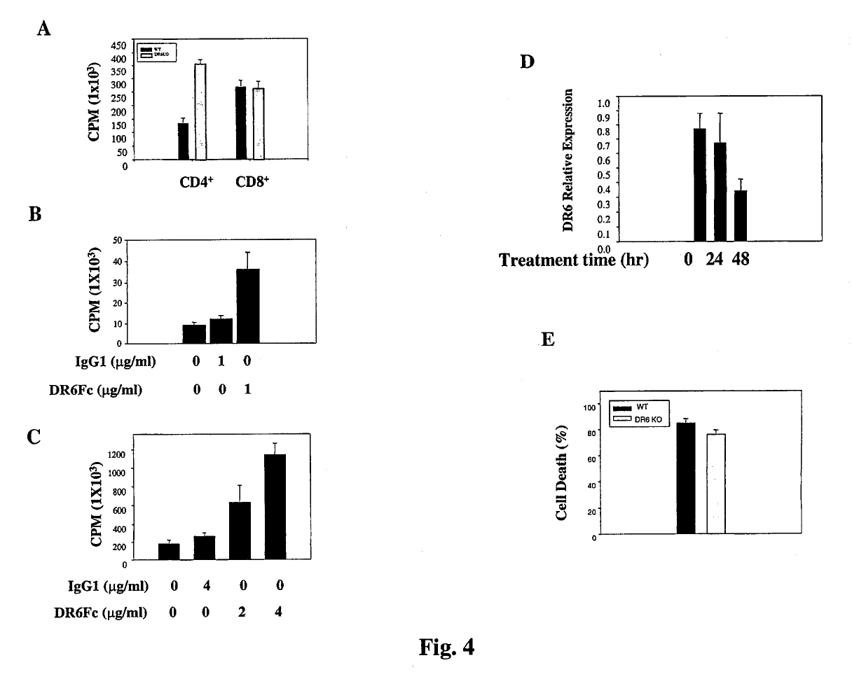

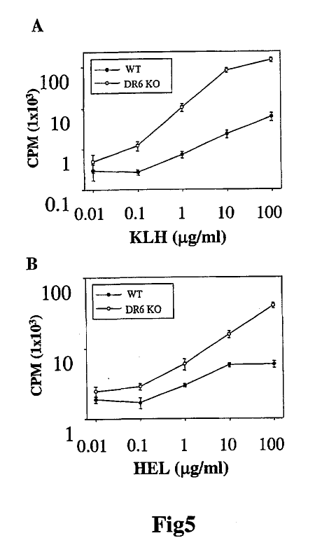

Applicants have shown that a DR6 deficiency in mice leads to hyperproliferation of CD4⁺ T cells in response to TCR stimulation and/or protein antigen challenges as well as enhanced Th2 cytokine expression. More specifically, the absence of DR6 resulted in enhanced CD4⁺ T cell growth in a CD28-independent manner in vitro. Furthermore, under in vitro conditions, mature peripheral T cells from DR6 deficient mice displayed a significantly stronger CD4⁺ T cell proliferative response than cells from wild type littermates upon activation with suboptimal concentrations of anti-TCR or anti-TCR combined with anti-CD28 costimulation, whereas CD8⁺ T cell growth was unaffected. Although DR6 is normally expressed in both CD4⁺ and CD8⁺ T cells (highest expression levels are present in resting T cells and DR6 expression level was observed to be dramatically reduced after 48 hr stimulation; see FIG. 4D), the studies disclosed herein showed DR6 deficiency to have profound effects only on CD4⁺ T cells and related mechanisms. No difference of cell surface expression of TCR/CD3 complex was observed between wild type and DR6 deficient T cells either before or after stimulation (data not shown). Furthermore, a combination of ionomycin and PMA also enhanced DR6 deficient CD4⁺ T-cell proliferation suggesting that the hyperproliferative state might be the result of DR6 actions on downstream TCR signaling or via regulation of other key growth regulating molecules normally present on T cells (data not shown).

Signaling through the IL-2 receptor (CD25) plays a pivotal role for T cell growth. Noteworthy then, is Applicant's observation that IL-2 receptor and costimulatory receptor (CD28) are dramatically upregulated in activated DR6 deficient T cells compared with wild type T cells whereas inhibitory receptor (CTLA4) is significantly down-regulated. Furthermore, DR6 deficient T cells showed augmented cell proliferation and cell cycle progression in response to exogenous IL-2 (data not shown) suggesting that the enhanced T cell proliferation in response to plate-bound antibodies, was at least partially, due to the increased expression of IL-2 receptor whereas the increased CD28 and reduced CTLA-4 expression resulted in the greater T cell response when APCs deliver the T cell activating signals, e.g. in vivo foreign antigen challenge. Even so, CD4⁺ hyperproliferation would also result if activation induced cell death was lessened as a result of DR6 deficiency. In fact, an in vivo study by Pan et al., indicated that DR6 could induce apoptosis in some cell types. However, no difference in activation induced cell death was observed between wild type and DR6 deficient T cells (FIG. 4E). Thus, DR6 seems to function more like a regulatory receptor rather than as an apoptosis modulator for TCR-mediated signaling.

CD28 is one of the most critical costimulatory molecules and also plays an important role in stimulating T cell proliferation and Th2 cell differentiation (Lenchow et al., CD28/B7 regulation of Th1 and Th2 subsets in the development of autoimmune diabetes, Immunity 5:285-293 (1996); Khattri et al., 1999). In contrast, CTLA-4, which is homologous to CD28, binds to the same ligand but delivers a negative signal to T cells that leads to the inhibition of T cell proliferation (Fallarino et al., B7-1 engegement of cytotoxic T lymphocyte antigen 4 inhibit T cell activation in the absence of CD28, J. Exp. Med. 188:205-210 (1998); Oosterwegel et al., The role of CTLA-4 in regulating Th2 differentiation. J. Immunol., 163:2634-2639 (1999)). Additionally, CTLA-4 costimulation has been reported to induce differentiation of naive CD4⁺ T cells towards Th1 differentiation by affecting IL-4 production (Oosterwegel et al., 1999; Nariuchi et al., 2000). Furthermore, the CD4⁺ T cell subset present in CTLA-4.deficient mice displayed predominantly Th2 responses and secreted high levels of IL-4 and IL-5 upon TCR stimulation. Accordingly, then decreased CTLA-4 expression and/or increased CD28 expression could account for the increases in both T cell proliferation and Th2 differentiation observed in DR6 deficient mice. Further supporting Applicant's conclusion that DR6 modulates signals generated through the TCR/CD28/CTLA-4 pathway are recent studies demonstrating that CTLA-4 deficient mice have a dramatic accumulation of T cells in lymph nodes and these T cells exhibited an activated phenotype (Madelbrot et al., 1999). Although, naive DR6 deficient mice did not demonstrate any significant difference in the numbers of thymus and peripheral T cells as compared to wild-type mice, after KLH challenge T cells from draining lymph nodes of DR6 deficient mice were about three times more abundant than those of their wild type littermates (data not shown). This observation is also consistent with the observation that CTLA-4 is down regulated only after T cells are activated (data not shown).

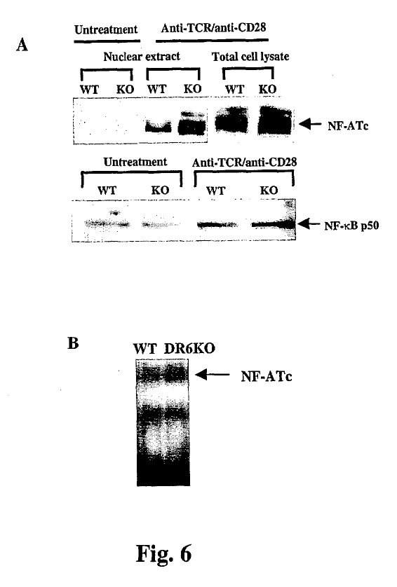

It is generally accepted that immune system is well coordinated by an interacting network of transcription factors. One of the most important of these transcription factors, NF-ATc, has been demonstrated to play a critical role in promoting peripheral T lymphocyte proliferation and Th2 cell differentiation. NF-ATc is also necessary for optimal IL-4 gene expression which is essential for Th2 cell polarization (Yoshida et al., 1998; Ranger et al., 1998a; Kuo et al., 1999; Yoshida et al., 1998). Applicants have discovered that in activated DR6 deficient CD4⁺ T cells the nuclear level of NF-ATc is significantly increased. Furthermore, the accumulated NF-ATc appeared to be functional as evidenced by the enhanced DNA binding activity detected (FIG. 6B). Interestingly, NF-ATc is also involved in upregulating CD25 expression in activated T cells (Schuh et al., The interleukin 2 receptor A chain/CD25 promoter is a target for nuclear factor of activated T cells, J. Exp. Med. 188:1369-1373 (1998)). However, in DR6 deficient T cells nuclear levels of NF-ATp exhibited no significant difference as compared levels from wild-type mice. NF-ATp is also a NFAT family member but it is known to be a negative regulator for T cell proliferation and Th2 cell differentiation (Ranger et al., Inhibitory function of two NFAT family members in lymphoid homeostasis and Th2 development, Immunity 9:627-635 (1998b)). The increased nuclear level of NF-ATc in the activated DR6 deficient T cells is likely responsible for the enhanced Th2 cytokine expression. The data presented herein provides the first evidence to suggest that DR6 is an important regulatory factor involved in controlling TCR-mediated T cell differentiation through a NF-ATc pathway.

NF-ATc is believed to be involved in Jnk signaling, whereas Jnk1/Jnk2 deficient T cells showed higher nuclear levels of NF-ATc and produce increased Th2 cytokines (Dong et al., Defective T cell differentiation in the absence of Jnk1, Science 282:2092-2095 (1998); Chow et al., c-Jun NH2-terminal kinase inhibits targeting of the protein phosphatase calcineurin to NF-ATc, Mol. Cell. Biol. 20:5227-5234 (2000); Dong et al., JNK is required for effector T-cell function but not for T-cell activation, Nature 405:91-94 (2000)). Therefore, absence of DR6 may, to some extent, reverse the inhibitory effect of Jnk on calcineurin to NF-ATc and promote nuclear translocation of NF-ATc.

Applicants also evaluated the regulatory function of DR6 in T cell responses in vivo using a well-characterized mouse model of GVHD (Via, C. S., Sharron, S. O., and Shearer, G. M., J. Immunol., 139:1840 (1987)). In this model, the recipients of DR6 deficient T cells showed earlier signs of GVHD and had significantly enhanced mortality and morbidity compared with the recipients of wild type T cells and T cell depleted splenocytes (TCD) from wild type mice. T cells from DR6 deficient mice engrafted, activated, and expanded in the host at dramatically faster speed compared with the wild type T cells and TCD. DR6 deficient T cells became cytolytic at significantly earlier time point and destroyed host B cells and double-positive thymocytes. Histology analysis showed intestinal lesions in the recipient of DR6 deficient T cells, and TUNEL assays also indicated significantly higher apoptosis in the intestine from the recipient of DR6 deficient T cells. Thus, DR6 is the first of the identified TNFR family members proven to be a negative regulator of T cell responsiveness to alloantigens.

Thus, a method of inhibiting the alloreactivity of T cells comprises administering at least one DR6 agonist to T cells in vitro or in vivo. Similarly, the present invention provides a method for treating or preventing autoimmunity, GVHD, T-cell mediated cytotoxicity, inflammatory bowel diseases, T-cell mediated inflammatory diseases, apoptosis, and/or at least one condition or symptom related thereto, in a mammal that comprise administering to said mammal a therapeutically effective amount of a pharmaceutical composition comprising at least one DR6 agonist. Preferably, the DR6 agonist is an agonistic anti-DR6 antibody. More preferably the DR6 agonist is a small molecule. Most preferably, the DR6 agonist is a naturally occurring ligand of DR6. Preferably, the administration of the DR6 agonist to the mammal is subsequent to the mammal having a bone marrow and/or solid organ transplantation.

IgE and eosinophil are two critical factors for eliciting allergic reactions and/or allergic autoimmune diseases such as asthma, atopy, and eczema. Th2 cytokines, especially IL-4 and IL-5, are critical for IgE production and eosinophil growth and differentiation. Since DR6 is a negative regulator of Th2 cell differentiation, a DR6 agonist would also be useful in reducing Th2 cell differentiation and/or Th2 cytokine production. Preferably such a use would be intended to treat, prevent, or delay onset and/or the escalation or progression of allergic reactions and/or allergic autoimmune diseases such as asthma, atopy, and/or eczema in a mammal. Therefore, the present invention also provides a method for treating or preventing asthma, allergy, atopy, eczema, and/or at least one condition or symptom related thereto, in a mammal that comprises administering to said mammal a therapeutically effective amount of a pharmaceutical composition comprising at least one DR6 agonist.

Another embodiment of the present invention provides a method of treating or preventing immunodeficiency, cancer, bacterial or viral infection, and/or at least one condition or symptom related thereto, in a mammal that comprises administering to said mammal a therapeutically effective amount of a pharmaceutical composition comprising at least one DR6 antagonist. Preferably, the DR6 antagonist is a small molecule. Alternatively, the DR6 antagonist is an antagonistic anti-DR6 human antibody. Even more preferably, the DR6 antagonist comprises a soluble form of DR6 (sDR6). Most preferably, the sDR6 comprises a polypeptide as shown from amino acid 42 through 350 of SEQ ID NO:2.

It is also an object of this invention to provide methods for enhancing cell mediated immunity in a mammal that comprise administering a therapeutically effective amount of a pharmaceutical composition comprising at least one DR6 antagonist. Preferably, the DR6 antagonist is a small molecule. Alternatively, the DR6 antagonist is an antagonistic anti-DR6 human antibody. Even more preferably, the DR6 antagonist comprises a sDR6. Most preferably, the sDR6 a polypeptide as shown from amino acid 42 through 350 of SEQ ID NO:2.

It is also an object of this invention to provide methods for inhibiting cell mediated immunity in a mammal that comprise administering a therapeutically effective amount of a pharmaceutical composition comprising at least one DR6 agonist. Preferably the DR6 agonist is an agonistic anti-DR6 antibody. More preferably the DR6 agonist is an agonistic anti-DR6 human antibody. Even more preferably the DR6 agonist is a small molecule. Most preferably a natural occurring ligand of DR6.

The data disclosed herein also support the administration of DR6 antagonists as an adjuvant for a mammal having a weakened immune system or a mammal otherwise in need of a bolstered immune system. Preferably such administration is intended to treat or prevent an immunodeficiency, cancer, complications from infection, and/or at least one condition or symptom related thereto, in a mammal.

The invention further provides for the use of a DR6 agonist or DR6 antagonist in the manufacture of a medicament for treating or preventing at least one symptom associated with immunodeficiency, cancer, bacterial or viral infection, complications of bacterial or viral infection, autoimmunity, GVHD, T-cell mediated cytotoxicity, inflammatory bowel diseases, T-cell mediated inflammatory diseases, apoptosis, asthma, allergy, atopy, and/or eczema in a mammal.

DR6 agonists or DR6 antagonists intended for administration to a mammal can be formulated into pharmaceutically acceptable compositions for preventing or treating DR6 associated conditions or diseases. Such formulations can be dosed in a fashion consistent with good medical practice, taking into account the clinical condition of the individual mammal (especially the side effects of treatment, the site of delivery of the composition, the method of administration, the scheduling of administration, and other factors known to practitioners.

The DR6 encoding polynucleotide as shown in SEQ ID NO:1 comprises a single large open reading frame. However, depending upon the starting point of translation, a polypeptide of 655 amino acids or 631 amino acids is predicted. The encoding nucleotide sequence of 1,968 nucleotide base pairs (including the stop codon) of SEQ ID NO:1 encodes a protein of 655 amino acid residues (SEQ ID NO:2). However, the second methionine may actually be the start of translation. In this case the encoding nucleotide sequence of SEQ ID NO:1 would encode a smaller 631 amino acid residue protein (SEQ ID NO:3). The difference in the encoded DR6 genes resides in the length of secretory leader sequence. The structure shown in SEQ ID NO:2, shows a 655 amino acid protein containing a 41 amino acid signal peptide (i.e., residues 1-41 of SEQ ID NO:2 (see Pan et al., supra)). Alternatively, a 631 amino acid protein is expressed containing a 17 amino acid signal peptide (SEQ ID NO:3). DNA sequences which have been engineered to start at the second methionine (as exemplified in Example 12) result in a molecule that is both secreted and processed. The isolated product corresponds to a mature protein of about 614 amino acids. It is likely that the polypeptides as shown in SEQ ID NO:2 and SEQ ID NO:3 would both result in a mature 614 amino acid polypeptide upon expression in mammalian cells.

One of the essential structural motifs found in the extracellular domain thought to be important for ligand binding are the cyteine rich motifs. Four cysteine rich motifs in the N-terminal domain, which are represented in a variety of related proteins, and which can form internal disulfide bonds, span from about amino acid residue 67 to about amino acid 211 of SEQ ID NO:2 (corresponding to about amino acid 43 to about amino acid 187 of SEQ ID NO:3). The cysteine rich motifs are part of the extracellular domain involved in binding ligands. The extracellular domain of SEQ ID NO:2 is contained within the region from about amino acids 42 to about 350. Functional extracellular domains can be produced comprising about amino acid 42 to about amino acids 211 through 214 of SEQ ID NO:2.

The methods of the present invention may utilize a sDR6 polypeptide as defined herein (e.g., amino acid 42-350 of SEQ ID NO:2) or a functional fragment and/or functional analog thereof. Functional fragments and/or analogs of the sDR6 polypeptides disclosed herein (e.g., amino acids 350-350 of SEQ ID NO:2) may be generated by deletion, insertion, or substitution of one or more amino acid residues of a sDR6 polypeptide as defined herein. Functional fragments and/or functional analogs of a sDR6 as defined herein also may be used in the methods of the present invention provided that the sDR6 fragment and/or analog retains the ability to bind a DR6 ligand and it can compete with DR6 for binding to DR6 ligands. Modifications of the amino acid sequence can generally be made in accordance with the substitutions provided in Table 1. The extracellular domain sequences of the sDR6 may optionally contain additional DR6 sequences or may be fused to an Ig constant region.

Skilled artisans will recognize that the polypeptides utilized in the methods of the present invention can be synthesized by a number of different methods, such as chemical methods well known in the art, including solid phase peptide synthesis or recombinant methods. Both methods are described in U.S. Pat. No. 4,617,149, incorporated herein by reference.

The principles of solid phase chemical synthesis of polypeptides are well known in the art and may be found in general texts in the area (see, e.g., H. Dugas and C. Penney, Bioorganic Chemistry (1981), Springer-Verlag, New York, 54-92). For example, peptides may be synthesized by solid-phase methodology utilizing an Applied Biosystems 430A peptide synthesizer (Applied Biosystems, Foster City, Calif.) and synthesis cycles supplied by Applied Biosystems.

The polypeptides utilized in the methods of the present invention can also be produced by recombinant DNA methods known in the art. Recombinant methods are preferred if a high yield is desired. Expression of the useful polypeptides can be carried out in a variety of suitable host cells, well known to those skilled in the art. For this purpose, the sDR6 constructs are introduced into a host cell by any suitable means, well known to those skilled in the art.

The basic steps in the recombinant production of polypeptides are:

a) constructing a recombinant, synthetic or semi-synthetic polypeptide encoding DNA;

b) integrating said DNA into an expression vector in a manner suitable for expressing the protein;

c) transforming or otherwise introducing said vector into an appropriate eukaryotic or prokaryotic host cell forming a recombinant host cell;

d) culturing said recombinant host cell in a manner to express the polypeptide; and

e) recovering and substantially purifying the polypeptide by any suitable means well known to those skilled in the art.

Methods of producing useful polynucleotides and polypeptides also include routes where direct chemical synthetic procedures are employed, and as well as products produced by recombinant techniques from a eukaryotic host, including, for example, yeast, higher plant, insect and mammalian cells. Depending upon the host employed in a recombinant production procedure, the polypeptides used in the methods of the present invention can be glycosylated or can be non-glycosylated. Additionally, the sequence of a polypeptide useful in the methods of the present invention may optionally include one or more conservative amino acid substitutions. Preferred polypeptides are glycosylated as would occur in eukaryotic hosts. In addition, the polypeptides molecules used in the methods of the present invention can also include an initial modified methionine residue, in some cases as a result of host-mediated processes. Such methods are described in many standard laboratory manuals, such as Sambrook, supra, Chapters 17.37-17.42; Ausubel, supra, Chapters 10, 12, 13, 16, 18 and 20, entirely incorporated herein by reference.

Fragments of proteins used in the methods of the present invention may be generated by any number of suitable techniques, including chemical synthesis. For instance constant regions of immunoglobulins can be obtained by papain digestion of antibodies. Such proteolytic digestion of, for example, SEQ ID NO:4 can produce the Ig constant region which then can be covalently linked to the extracellular domain of sDR6. Alternatively, recombinant DNA mutagenesis techniques can provide useful polynucleotides or polypeptides (see generally. e.g. K. Struhl, “Reverse biochemistry: Methods and applications for synthesizing yeast proteins in vitro,” Meth. Enzymol. 194:520-535). For example, a nested set of deletion mutations are introduced into the sDR6 DNA such that varying amounts of the protein coding region are deleted, either from the amino terminal end, or from the carboxyl end of the protein molecule. Further, additional changes or additions to the molecule can be made. This method can also be used to create internal fragments of the intact protein in which both the carboxyl and/or amino terminal ends are removed. Several appropriate nucleases can be used to create such deletions, for example Bal31l, or in the case of a single stranded nucleic acid compound, mung bean nuclease. For simplicity, it is preferred that the intact DR6 gene be cloned into a single-stranded cloning vector, such as bacteriophage M13, or equivalent. If desired, the resulting gene deletion fragments can be subcloned into any suitable vector for propagation and expression of said fragments in any suitable host cell.

The present invention also contemplates use of fragments or analogs of a sDR6 polypeptide disclosed wherein said fragments retain ability to bind a DR6 ligand. As used herein, the phrase “functional fragments” includes fragments whether or not fused to additional sequences, that retain and exhibit, under appropriate conditions, measurable ligand binding activity, i.e., the sDR6 fragment is able to effectly compete for the binding of a natural ligand to a functioning cell receptor. Accordingly, in one embodiment, the invention features treating or preventing the T cell associated disorders described herein in a mammal by administering to the mammal a therapeutically effective amount of a pharmaceutical composition that comprises a sDR6 or a functional fragment thereof.

Functional fragments of the proteins utilized in the methods disclosed herein may be produced as described above, preferably using cloning techniques to engineer smaller versions of the a functioning sDR6, lacking sequence from the 5′ end, the 3′ end, from both ends, or from an internal site.

A functional sDR6 can additionally be fused to a marker protein or an epitope tag. Such fusions include, but are not limited to, fusions to an enzyme, fluorescent protein, or luminescent protein which provide a marker function; or fusions to any amino acid sequence which can be employed for purification of the polypeptide or a proprotein sequence.

Methods of constructing fusion proteins (chimeras) composed of the binding domain of one protein and the constant region of an immunglobulin (herein designated as “sDR6-Ig”) are generally known in the art. For example, chimeras containing the Fc region of human IgG and the binding region of other protein receptors are known in the art for chimeric antibodies. sDR6-Ig structures of the present invention can be constructed using methods similar to the construction of chimeric antibodies. In chimeric antibody construction the variable domain of one antibody of one species is substituted for the variable domain of another species (see EP 0125023; EP 173,494; Munro et al., Nature, 312:597 (1984); Neuberger et al. Nature, 312:604-608 (1984); Sharon et al., Nature, 309:364-367; Morrison et al., Ann. Rev. Immunol., 2:239-256 (1984); Morrison et al., 1985; Boulianne et al., Nature, 312:643-646 (1984); Capon et al., Nature, 337:525-531 (1989); Traunecker et al., Nature, 339:68-70 (1989)). Here, a functional extracellular domain of sDR6 is substituted for the variable domain of the recipient antibody structure.

Generally, methods for constructing the polypeptide structures used in the methods of the present invention would include use of recombinant DNA technology. For instance, the DNA encoding a functional extracellular domain, optionally with additional domains or segments of the DR6 or as fusions with an Ig constant region can be obtained by PCR or by restriction enzyme cleavage at or near the 5′ end of the mature DR6 (where a different leader is contemplated) and with a restriction enzyme cleavage at or proximal to the 3′ end of the DNA that is to be joined to the immunoglobulin-like domain (optionally the joined regions may include a linker region). This DNA fragment is readily inserted proximal to DNA encoding an immunoglobulin light or heavy chain constant region and, if necessary, the resulting construct is tailored by mutagenesis, to insert, delete, or change the codon sequence. Preferably, the selected immunoglobulin region is a human immunoglobulin region when the chimeric molecule is intended for in vivo therapy for humans. Most preferably, the selected immunoglobulin region is an IgG region. DNA encoding immunoglobulin light or heavy chain constant regions are known or readily available from cDNA libraries or can be synthesized (see for example Adams et al., Biochemistry, 19:2711-2719 (1980); Gough et al., Biochemistry, 19:2702-2710 (1980); Dolby et al., 1980; Rice et al., Proc. Nat'l. Acad. Sci., 79:7862-7865 (1982); Falkner et al., Nature, 298:286-288 (1982); and Morrison et al., Ann. Rev. Immunol., 2:239-256 (1984)). Other teachings of preparing chimeric molecules are known from the preparation of immunoadhesion chimerics, such as CD4-Ig (Capon et al., Nature, 337:525-531 (1989); Byrn et al., Nature, 344:667 (1990)) and TNFR chimerics, such as TNFR-IgG (Ashkenazi, et al., Proc. Natl. Acad. Sci., 88:10535-10539 (1991); Peppel et al., J. Cell. Biochem. Supp. 15F-P439:118 (1991)).

Protein Purification

Generally, polypeptides useful in the methods of the present invention may be produced recombinantly. Once expressed they can be isolated from the cells by applying standard protein isolation techniques to the lysates or purified from the media. The monitoring of the purification process can be accomplished by using standard Western blot techniques or radioimmunoassays or other standard immunoassay techniques.

Polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, size exclusion chromatography, and lectin chromatography. Preferably, high performance liquid chromatography (“HPLC”), cation exchange chromatography, affinity chromatography, size exclusion chromatography, or combinations thereof, are employed for purification. Particular methods of using protein A or G chromatography for purification are known in the art and are particularly applicable where the polypeptide contains immunoglobulin Fc region. Protein A and G binds the Fc regions of IgG antibodies, and therefore makes a convenient tool for the purification of polypeptides containing the IgG region. sDR6 purification is meant to include purified parts of the chimeric (the extracellular region and the immunoglobulin constant region) that are purified separately and then combined by disulfide bonding, cross-linking or the like.

The purification of polypeptides can be accomplished by a number of special techniques known in the art (Kwon, et al., J. Biol. Chem.: 272:14272-14276 (1997); Emery, J. G., et al., J. Biol. Chem., 273:14363-14367 (1998); Harrop et al., J. Biol. Chem., 273(42):27548-27556 (1998); Harrop et al., J. Immunol.: 161:1786-1794 (1998)) that take particular advantage of structural and functional features of these molecules. Further, a number of advantageous protein sequences can be incorporated into the polypeptide produced, such as factor Xa cleavage sites or an HIS tag sequence or the incorporation of specific epitopes, as is known in the art.

Gene Isolation Procedures

Cloning of DR6 cDNAS and knowledge of the cDNA structures have been reported by several groups (Pan et al., FEBS Letters 431:351-356 (1998), and also appears in the GeneBank database as accession no. AF068868; TR7 reported in EP 0869 79 A1; DR6 homologs, as well as a 253 amino acid natural soluble variant is reported in WO99/1566). Those skilled in the art will recognize that the polypeptides utilized in the methods of the present invention can be obtained by a plurality of recombinant DNA techniques including, for example, hybridization, polymerase chain reaction (PCR) amplification, or de novo DNA synthesis. (See e.g., T. Maniatis et al., Molecular Cloning: A Laboratory Manual, 2d Ed. Chap. 14 (1989)).

Methods for constructing cDNA libraries in a suitable vector such as a plasmid or phage for propagation in prokaryotic or eukaryotic cells are well known to those skilled in the art (See, e.g., Maniatis et al., supra). Suitable cloning vectors are well known and are widely available.

The DR6 gene can be isolated from any tissue in which said DR6 is expressed. A suitable tissue can be selected from heart, brain, placenta, pancreas, lymph node, thymus, and prostate. In one method, mRNA is isolated from a suitable tissue, and first strand cDNA synthesis is carried out. A second round of DNA synthesis can be carried out for the production of the second strand. If desired, the double-stranded cDNA can be cloned into any suitable vector, for example, a plasmid, thereby forming a cDNA library. Oligonucleotide primers targeted to any suitable region can be used for PCR amplification of DR6 sequences (see, e.g. PCR Protocols: A Guide to Method and Application, Ed. M. Innis et al., Academic Press (1990)). The PCR amplification comprises template DNA, suitable enzymes, primers, and buffers, and is conveniently carried out in a DNA Thermal Cycler (Perkin Elmer Cetus, Norwalk, Conn.). A positive result is determined by detecting an appropriately-sized DNA fragment following agarose gel electrophoresis.

Expressing Recombinant Proteins in Host Cells

Prokaryotes may be employed in the production of recombinant proteins. For example, the Escherichia coli K12 strain 294 (ATCC No. 31446) is particularly useful for the prokaryotic expression of foreign proteins. Other strains of E. coli, bacilli such as Bacillus subtilis, enterobacteriaceae such as Salmonella typhimurium or Serratia marcescans, various Pseudomonas species and other bacteria, such as Streptomyces, may also be employed as host cells in the cloning and expression of the recombinant proteins of this invention.

Promoter sequences suitable for driving the expression of genes in prokaryotes include β-lactamase (e.g., vector pGX2907, ATCC 39344, contains a replicon and β-lactamase gene), lactose systems (Chang et al., Nature (London), 275:615 (1978); Goeddel et al., Nature (London), 281:544 (1979)), alkaline phosphatase, and the tryptophan (trp) promoter system (vector PATH1 (ATCC 37695)), which is designed to facilitate expression of an open reading frame as a trpE fusion protein under the control of the trp promoter. Hybrid promoters such as the tac promoter (isolatable from plasmid pDR540, ATCC-37282) are also suitable. Still other bacterial promoters, whose nucleotide sequences are generally known, may be ligated to DNA encoding the protein of the instant invention, using linkers or adapters to supply any required restriction sites. Promoters for use in bacterial systems also will contain a Shine-Dalgarno sequence operably linked to the DNA encoding the desired polypeptides. These examples are illustrative rather than limiting.

The proteins used in the methods of the present invention may be synthesized either by direct expression or as a fusion protein comprising the protein of interest as a translational fusion with another protein or peptide which may be removed by enzymatic or chemical cleavage. It is often observed in the production of certain peptides in recombinant systems that expression as containing other desired sequences prolongs the lifespan, increases the yield of the desired peptide, or provides a convenient means of isolating the protein. This is particularly relevant when expressing mammalian proteins in prokaryotic hosts. A variety of peptidases (e.g., enterokinase and thrombin) which cleave a polypeptide at specific sites or digest the peptides from the amino or carboxy termini (e.g., diaminopeptidase) of the peptide chain are known. Furthermore, particular chemicals (e.g., cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites (see, e.g., P. Carter, “Site Specific Proteolysis of Fusion Proteins”, Chapter 13, in Protein Purification: From Molecular Mechanisms to Large Scale Processes, American Chemical Society, Washington, D.C. (1990)).

The choice of a particular host cell depends to some extent on the particular expression vector used. Exemplary mammalian host cells suitable for producing the polypeptides used in the methods of the present invention include 293 (e.g., ATCC CCL 1573), HepG-2 (ATCC HB 8065), CV-1 (ATCC CCL 70), LC-MK2 (ATCC CCL 7.1), 3T3 (ATCC CCL 92), CHO-K1 (ATCC CCL 61), HeLa (ATCC CCL 2), RPMI8226 (ATCC CCL 155), H4IIEC3 (ATCC CCL 1600), C127I (ATCC CCL 1616), HS-Sultan (ATCC CCL 1484), and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSV2-dhfr, pSV2-hyg, and pSV2-β-globin, in which the SV40 promoter drives transcription of an inserted gene. These vectors are widely available from sources such as the American Type Culture Collection (ATCC), 12301 Parklawn Drive, Rockville, Md., 20852, or the National Center for Agricultural Utilization Research, 1815 North University Street, Peoria, Ill. 61604-39999.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes.

Plasmid pRSVcat (ATCC 37152) comprises portions of a long terminal repeat of the Rous Sarcoma virus, a virus known to infect chickens and other host cells. This long terminal repeat contains a promoter which is suitable for use in the vectors of this invention. (H. Gorman et al., Proc. Nat. Acad. Sci. (USA), 79, 6777 (1982)). The plasmid pMSVi (NRRL B-15929) comprises the long terminal repeats of the Murine Sarcoma virus, a virus known to infect mouse and other host cells. The mouse metallothionein promoter has also been well characterized for use in eukaryotic host cells and is suitable for use in the present invention. This promoter is present in the plasmid pdBPV-MMTneo (ATCC 37224) which can serve as the starting material for the construction of other plasmids of the present invention.

Transfection of mammalian cells with vectors can be performed by a plurality of well known processes including, but not limited to, protoplast fusion, calcium phosphate co-precipitation, electroporation and the like. See, e.g., Maniatis et al., supra.

Some viruses also make appropriate vectors. Examples include the adenoviruses, the adeno-associated viruses, the vaccinia virus, the herpes viruses, the baculoviruses, and the Rous Sarcoma virus, as described in U.S. Pat. No. 4,775,624, incorporated herein by reference.

Eukaryotic microorganisms such as yeast and other fungi are also suitable host cells. The yeast Saccharomyces cerevisiae is the preferred eukaryotic microorganism. Other yeasts such as Kluyveromyces lactis and Pichia pastoris are also suitable. For expression in Saccharomyces, the plasmid YRp7 (ATCC-40053), for example, may be used. See, e.g., L. Stinchcomb et al., Nature, 282, 39 (1979); J. Kingsman et al., Gene, 7, 141 (1979); S. Tschemper et al., Gene, 10, 157 (1980). Plasmid YRp7 contains the TRP1 gene which provides a selectable marker for use in a trp1 auxotrophic mutant.

Purification of Recombinantly-Produced Proteins

An expression vector carrying the cloned polypeptide encoding polynucleotide, or fragment thereof, is transformed or transfected into a suitable host cell using standard methods. Cells that contain the vector are propagated under conditions suitable for expression of the recombinant protein. For example, if the recombinant gene has been placed under the control of an inducible promoter, suitable growth conditions would incorporate the appropriate inducer. The recombinantly-produced protein may be purified from cellular extracts of transformed cells by any suitable means.

In a preferred process for protein purification, the polypeptide encoding polynucleotide sequences are modified at the 5′ end to encode for several histidine residues at the amino terminus of the polypeptide. This “histidine tag” enables a single-step protein purification method referred to as “immobilized metal ion affinity chromatography” (IMAC), essentially as described in U.S. Pat. No. 4,569,794, which hereby is incorporated by reference. The IMAC method enables rapid isolation of substantially pure recombinant protein starting from a crude extract, of cells that express a modified recombinant protein, as described above.

Production of Antibodies

The production of antibodies, both monoclonal and polyclonal, in animals, especially mice, is well known in the art (see, e.g., C. Milstein, Handbook of Experimental Immunology, (Blackwell Scientific Pub., (1986); J. Goding, Monoclonal Antibodies: Principles and Practice, Academic Press, (1983)). For the production of monoclonal antibodies, the basic process begins with injecting a mouse, or other suitable animal, with an immunogen. The mouse is subsequently sacrificed and cells taken from its spleen are fused with myeloma cells, resulting in a hybridoma that reproduces in vitro. The population of hybridomas is screened to isolate individual clones, each of which secretes a single antibody species, specific for the immunogen. Each antibody obtained in this way is the clonal product of a single B cell.

Chimeric antibodies are described in U.S. Pat. No. 4,816,567, the entire contents of which are herein incorporated by reference. This reference discloses methods and vectors for the preparation of chimeric antibodies. An alternative approach is provided in U.S. Pat. No. 4,816,397, the entire contents of which are herein incorporated by reference. This patent teaches co-expression of the heavy and light chains of an antibody in the same host cell.

The approach of U.S. Pat. 4,816,397 has been further refined in European Patent Publication No. 0239400. The teachings of this European patent publication are a preferred format for genetic engineering of monoclonal antibodies. In this technology the complementarity determining regions (CDRs) of a human antibody are replaced with the CDRs of a murine monoclonal antibody, thereby converting the specificity of the human antibody to the specificity of a murine antibody.

Single chain antibodies and libraries thereof are yet another variety of genetically engineered antibody technology that is well known in the art (see, e.g, R. E. Bird, et al., Science 242:423-426 (1988) and PCT Publications WO 88/01649, WO 90/14430, and WO 91/10737). Single chain antibody technology involves covalently joining the binding regions of heavy and light chains to generate a single polypeptide chain. The binding specificity of the intact antibody molecule is thereby reproduced on a single polypeptide chain.

The techniques for producing antibodies are well known to skilled artisans. (See e.g., A. M. Campbell, Monoclonal Antibody Technology: Laboratory Techniques in Biochemistry and Molecular Biology, Elsevier Science Publishers, Amsterdam (1984); Kohler and Milstein, Nature 256, 495-497 (1975); Monoclonal Antibodies: Principles & Applications Ed. J. R. Birch & E. S. Lennox, Wiley-Liss (1995)).

A protein used as an immunogen may be modified or administered in an adjuvant, by subcutaneous or intraperitoneal injection into, for example, a mouse or a rabbit. For the production of monoclonal antibodies, spleen cells from immunized animals are removed, fused with myeloma cells, such as SP2/0-Ag14 cells, and allowed to become monoclonal antibody producing hybridoma cells in the manner known to the skilled artisan. Hybridomas that secrete a desired antibody molecule can be screened by a variety of well known methods, for example ELISA assay, western blot analysis, or radioimmunoassay (Lutz et al., Exp. Cell Res. 175, 109-124 (1988); Monoclonal Antibodies: Principles & Applications Ed. J. R. Birch & E. S. Lennox, Wiley-Liss (1995)).

For some applications labeled antibodies are desirable. Procedures for labeling antibody molecules are widely known, including, for example, the use of radioisotopes, affinity labels such as biotin or avidin, enzymatic labels such as horseradish peroxidase, and fluorescent labels, such as FITC or rhodamine (See e.g., Enzyme-Mediated Immunoassay, Ed. T. Ngo, H. Lenhoff, Plenum Press (1985); Principles of Immunology and Immunodiagnostics, R. M. Aloisi, Lea & Febiger (1988)).

Anti-DR6 antibodies may be used in a screen to identify potential modulators of DR6. For example, in a competitive displacement assay, the antibody or compound to be tested is labeled by any suitable method. Competitive displacement of an antibody from an antibody-antigen complex by a test compound provides a method for identifying compounds that bind the sDR6.

Nucleic Acids

The methods of the present invention contemplate use of isolated nucleic acid sequences, including the polynucleotide having the sequence as shown in SEQ ID NO:1 and fragments thereof and polynucleotides complementary thereto. As skilled artisans will recognize, the amino acid compounds used in carrying out the methods of the present invention can be encoded by a multitude of different nucleic acid sequences, owing to the degeneracy of the genetic code.

Vectors

When preparing an expression vector the skilled artisan understands that there are many variables to be considered, for example, whether to use a constitutive or inducible promoter. The practitioner also understands that the amount of nucleic acid or protein to be produced dictates, in part, the selection of the expression system. Regarding promoter sequences, inducible promoters are preferred because they enable high level, regulatable expression of an operably-linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant protein. For example, a sequence encoding a signal peptide preceding the coding region of a gene is useful for directing the extra-cellular export of a resulting polypeptide.

One means to test the functionality of various DR6 agonists or DR6 antagonists in the methods of the present is provided by screening for those molecules that have the ability to bind to DR6 or a natural ligand of DR6 and/or to compete for the binding of a natural DR6 ligand. For example, methods for identifying or characterizing molecules as DR6 agonists or DR6 antagonists may comprise contacting at least one cell expressing DR6 with a candidate DR6 agonist or DR6 antagonist molecule and measuring a detectable change in one or more biological activities known to be associated with DR6 activation. Development of such methods for identifying or characterizing molecules as DR6 agonists or DR6 antagonists is within the skill of an ordinarily skilled artisan.

As a general proposition, the total pharmaceutically effective amount of a DR6 agonist or DR6 antagonist administered parentally per dose will be in the range of about 1 μg/kg/day to 10 mg/kg/day of body weight. However, as noted above, this will be subject to therapeutic discretion. Preferably, this dose is at least 0.001 mg/kg/day, or at least 0.01 mg/kg/day, or at least 0.10 mg/kg/day, or at least 1.0 mg/kg/day.

As a further proposition, if given continuously, the a DR6 agonist or DR6 antagonist is typically administered at a dose rate of about 0.1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appear to vary depending on the desired effect.

Pharmaceutical compositions containing a DR6 agonist or DR6 antagonist as described herein may be administered using a variety of modes that include, but are not limited to, oral, rectal, intra-cranial, parenteral, intracisternal, intrathecal, intravaginal, intraperitoneal, intratracheal, intrabroncho-pulmonary, topical, transdermal (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include, but are not limited to, intravenous, intramuscular, intraperitoneal, intrasternal, subcutaneous and intraarticular injection and infusion. Implants comprising a DR6 agonist or DR6 antagonist also can be used.

A DR6 agonist or DR6 antagonist is also suitably administered by sustained-release systems. Suitable examples of sustained-release compositions include semi-permeable polymer matrices, e.g., films, or microcapsules. Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate (Langer, R., et al., J. Biomed. Mater. Res. 15:167-277 (1981))), ethylene vinyl acetate (R. Langer et al., 1982) or poly-D-3-hydroxybutyric acid (EP 133,988).

Sustained-release DR6 agonist or DR6 antagonist compositions also include liposomally entrapped DR6 agonists or DR6 antagonists. Liposomes containing a DR6 agonist or DR6 antagonist are prepared by methods known per se (DE 3,218,121; Epstein et al., Proc. Natl. Acad. Sci. (USA) 82:3688-3692 (1985); Hwang et al., Proc. Natl. Acad. Sci. (USA) 77:4030-4034 (1980)); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal sDR6 polypeptide therapy.

For parenteral administration, in one embodiment, the a sDR6 polypeptide or DR6 antibody is formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting a DR6 agonist or DR6 antagonist uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

A sDR6 polypeptide or DR6 antibody is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of salts of the sDR6 polypeptide or DR6 antibody. sDR6 polypeptides or DR6 antibodies to be used for therapeutic administration must be sterile. Sterility is readily accomplished by filtration through sterile filtration membranes (e.g., 0.2 micron membranes). Therapeutic sDR6 polypeptide or DR6 antibody compositions generally are placed into a container having a sterile access port, for example, an intravenous solution bag or vial having a stopper pierceable by a hypodermic injection needle.

A sDR6 polypeptide or DR6 antibody intended for administration to a mammal ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous sDR6 polypeptide or DR6 antibody solution, and the resulting mixture is lyophilized. The infusion solution is prepared by reconstituting the lyophilized sDR6 polypeptide or DR6 antibody using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use, or sale for human administration. In addition, the polypeptides of the present invention may be employed in conjunction with other therapeutic compounds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present application, including definitions, will control. The following examples more fully describe the present invention. Those skilled in the art will recognize that the particular reagents, equipment, and procedures described are merely illustrative and are not intended to limit the present invention in any manner.

Materials

The various restriction enzymes disclosed and described herein are commercially available and the manner of use of said enzymes including reaction conditions, cofactors, and other requirements for activity are well known to one of ordinary skill in the art. Reaction conditions for particular enzymes were carried out according to the manufacturer's recommendation.

### EXAMPLE 1

### RT-PCR Amplification of a DR6 cDNA from mRNA

A DR6 encoding polynucleotide can be isolated by reverse transcriptase PCR (RT-PCR) using conventional methods known in the art. Total RNA from a tissue that expresses the DR6 gene, for example, heart, brain, spleen, liver, kidney, or lymph nodes is prepared using standard methods. First strand DR6 cDNA synthesis is achieved using a commercially available kit (SuperScript™ System; Life Technologies) by PCR in conjunction with specific primers directed at any suitable region of SEQ ID NO:1.

Amplification is carried out by adding to the first strand cDNA (dried under vacuum): 8 μl of 10× synthesis buffer (200 mM Tris-HCl, pH 8.4; 500 mM KCl, 25 mM MgCl2, 1 μg/ul BSA); 68 μl distilled water; 1 μl each of a 10 μM solution of each primer; and 1 μl Taq DNA polymerase (2 to 5 U/μl). The reaction is heated at 94° C. for 5 minutes to denature the RNA/cDNA hybrid. Then, 15 to 30 cycles of PCR amplification are performed using any suitable thermal cycle apparatus. The amplified sample may be analyzed by agarose gel electrophoresis to check for an appropriately-sized fragment.

### EXAMPLE 2

### Construction of Human sDR6-Fc

Recombinant construction of a sDR6-Fc fusion can be accomplished by fusing the extracellular domain of TFR6 with an Ig constant region. Essentially, the 5′ portion of the DR6 containing the leader sequence and the extracellular domain is amplified by polymerase chain reaction. The 5′ forward primer contains a Hind III site on the 5′ end of the primer and a 3′ Bgl II site at the 5′ end of the reverse primer to directionally orient the amplified fragment after digestion with the appropriate restriction enzymes. A Fc portion of human IgG1 can be PCR-amplified from ARH-77 (ATCC CRL-1621) cell RNA and cloned into the SmaI site of pGEM7 vector (Promega, Madison, Wis.). A suitable Fc fragment, including the CH2 and CH3 domain sequences is contained on a 5′-BglII/XhoI-3′ fragment. The HindIII/BgII extracellular domain fragment of DR6 can be inserted in-frame upstream of the human IgG1:Fc fragment. In-frame fusion of the two regions can be confirmed by sequencing. The DR6-Fc fragment can be released by digesting the plasmid with HINDIII/XhoI and cloned into an expression plasmid.

### EXAMPLE 3

### Expression of sDR6 in Mammalian Cells

A typical mammalian expression vector contains at least one promoter element, which mediates the initiation of transcription of mRNA, the polypeptide coding sequence, and signals required for the termination of transcription and polyadenylation of the transcript. Additionally, each mammalian expression vector may have enhancers, Kozak sequences and intervening sequences flanked by donor and acceptor sites for RNA splicing.

Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in providing sDR6 include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human HeLa, 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the desired DNA sequences for sDR6 can be expressed in stable cell lines that contain the DNA sequences for expressing each subunit once integrated into a chromosome(s). The co-transfection with a selectable marker such as dhfr, gpt, neomycin, or hygromycin allows the identification and isolation of the transfected cells as known in the art.

The sDR6 cDNA sequences harbored by the transfected cells can also be amplified to allow the expression of large amounts of the polypeptide encoded thereby. The DHFR (dihydrofolate reductase) marker is useful to develop cell lines that carry several hundred or even several thousand copies of the DNA sequences of interest. Another useful selection marker is the enzyme glutamine synthase (GS) (Murphy et al., Biochem. J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong promoter (LTR) of the Rous Sarcoma virus (Cullen et al., Molec. Cell. Biol. 5:438-447 (1985)) plus a fragment of the CMV-enhancer (Boshart et al., Cell 41:521-530 (1985)). Multiple cloning sites, e.g., with the restriction enzyme cleavage sites BamHI, XbaI and Asp718, facilitate the cloning of the sTNRF6 DNA sequences. The vectors contain, in addition to the 3′ intron, the polyadenylation and termination signal of the rat preproinsulin gene.

NIH T3T cells can be transfected with a PvuI linearized expression plasmid using the calcium phosphate coprecipitation method. Neomycin clones can be selected in 400 μg/ml G418 and selected clones expanded. Producing clones can be selected using an enzyme-linked immunoabsorbant assay with anti-human IgG1 and Northern analysis with a P32-labeled probe specific for polynucleotides encoding the DR6 extracellular domain. Similarly, clones producing the sDR6-Fc product can be produced in COS or CHO cells.

### EXAMPLE 4

### Cloning and Expression in COS or CHO Cells

An expression plasmid for a sDR6 is made by cloning a cDNA encoding a sDR6 into the expression vector pcDNAI/Amp or pcDNAIII (which can be obtained from Invitrogen, Inc.). The expression vector(s) pcDNAI/amp and pcDNA III contain: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) or HIS tag (see, e.g., Ausubel, et al., ed., Current Protocols in Molecular Biology, John Wiley and Sons, NY (1987-1999)) followed by a termination codon and polyadenylation signal arranged so that the cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide as has been previously described (Wilson et al., Cell 37:767-778 (1984)). The fusion of the HA tag to a sDR6 polypeptide, allows easy detection and recovery of the recombinant polypeptide with an antibody that recognizes the HA epitope. pcDNAIII contains, in addition, the selectable neomycin marker.

A cDNA fragment encoding a sDR6, is separately cloned into the polylinker region of the vector so that recombinant polypeptide expression is directed by the CMV promoter. Insertion into the vector is optionally with or without the HA epitope. The plasmid construction strategy is as follows. A sDR6 cDNA can be amplified using primers that contain convenient restriction sites. The PCR amplified sDR6 DNA fragment and the pcDNAI/Amp vector are digested with suitable restriction enzyme(s) and then the sDR6 DNA fragment is ligated to a digested vector. Each ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA for each subunit is isolated from resistant colonies and examined by restriction analysis or other means for the presence of sDR6 encoding fragment.

For expression of a sDR6, COS cells are co-transfected with an expression vector, as described above, using DEAE-DEXTRAN, as described, for instance, in Sambrook et al., Molecular Cloning: A Laboratory Manual, Cold Spring Laboratory Press, Cold Spring Harbor, N.Y. (1989). Cells are incubated under conditions for expression of sDR6.

A sDR6-HA polypeptide can be detected by radio-labeling and immunoprecipitation, using methods described in, for example, Harlow et al., Antibodies: A Laboratory Manual, 2nd Ed., Cold Spring Harbor Laboratory Press, Cold Spring Harbor, N.Y. (1988). To this end, two days after transfection, the cells are labeled by incubation in media containing 35S-cysteine for 8 hours. The cells and the media are collected, and the cells are washed and lysed with detergent-containing RIPA buffer: 150 mM NaCl, 1% NP-40, 0.1% SDS, 0.5% DOC, 50 mM TRIS, pH 7.5, as described by Wilson et al., Cell, 37:767-778 (1984). Proteins are precipitated from the cell lysate and from the culture media using an HA-specific monoclonal antibody. The precipitated protein is then analyzed by SDS-PAGE and autoradiography. An expression product of the expected size is seen in the cell lysate, which is not seen in negative controls.

The vector pC4 can be used for expression of sDR6. Plasmid pC4 is a derivative of the plasmid pSV2-DHFR (ATCC Accession No. 37146). The plasmid contains the mouse DHFR gene under control of the SV40 early promoter. Chinese hamster ovary (dhfr-) or other cells lacking dihydrofolate activity that are co-transfected with sDR6 plasmids can be selected by growing the cells in a selective medium (alpha minus MEM, Life Technologies) supplemented with the chemotherapeutic agent methotrexate (MTX). The amplification of the DHFR genes in cells resistant to methotrexate has been well documented (see, e.g., Alt et al., J. Biol. Chem., 253:1357-1370 (1978); Hamlin et al., Biophys. Acta 1097:107-143 (1990); and Page et al., Biotechnology 9:64-68 (1991)). Cells grown in increasing concentrations of MTX develop resistance to the drug by overproducing the target enzyme, DHFR, as a result of amplification of the DHFR gene. If DNA sequences are linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained which contain the amplified DNA sequences integrated into one or more chromosome(s) of the host cell.

Plasmid pC4 contains the strong promoter of the long terminal repeat (LTR) of the Rous Sarcoma virus (Cullen et al., Molec. Cell. Biol. 5:438-447 (1985) for expression of inserted gene sequences. PC4 additionally contains a fragment isolated from the enhancer of the immediate early gene of human cytomegalovirus (CMV) (Boshart et al., Cell 41:521-530 (1985)). Downstream of the promoter are BamHI, XbaI, and Asp718 restriction enzyme cleavage sites that allow integration of the DNA sequences. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express sDR6, in a regulated way in mammalian cells (Gossen et al., Proc. Natl. Acad. Sci. USA 89:5547-5551 (1982)). For the polyadenylation of the mRNA other signals, e.g., from the human growth hormone or globin genes, can be used as well. Stable cell lines carrying the DNA sequences of sDR6 integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel. The DNA sequence encoding the complete sDR6 sequence is amplified using PCR oligonucleotide primers corresponding to the 5′ and 3′ sequences of the gene. Non-limiting examples include 5′ and 3′ primers having nucleotides corresponding or complementary to a portion of the coding sDR6 according to methods known in the art.

The amplified fragment(s) are digested with suitable endonucleases and then purified again on a 1% agarose gel. The isolated fragments for each subunit and the dephosphorylated vector are then separately ligated with T4 DNA ligase. E. coli HB101 or XL-1 Blue cells are then separately transformed and bacteria are identified that contain the fragment (or fragments if the vector is adapted for expressing both alpha and beta subunits) inserted into plasmid pC4 using, for instance, restriction enzyme analysis.

Chinese hamster ovary (CHO) cells lacking an active DHFR gene are used for transfection. 5 μg of the expression plasmid(s) pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained which grow at a concentration of 100-200 mM. Expression of the desired product is analyzed, for instance, by SDS-PAGE and Western blot or by reverse phase HPLC analysis.

### EXAMPLE 5

### Expression of sDR6-Fc-rabbit in CHO Cells

The production, characterization, and use of sDR6:Fc-rabbit is produced using known techniques in the art but utilizing the rabbit Fc region as the fusion partner with the DR6 extracellular domain. Essentially, CHO-K1 cells are transfected with the sDR6Fc-rabbit using LipofectAMINE and maintained in F12 medium supplemented with 3% FBS. Medium is harvested and the sDR6:Fc is purified from the medium by Protein G-Sepharose chromatography.

### EXAMPLE 6

### Prokaryotic Expression Vectors for sDR6

An expression vector suitable for expressing sDR6 or fragment thereof in a variety of prokaryotic host cells, such as E. coli, is easily made. The vector contains an origin of replication (Ori), an ampicillin resistance gene (Amp) useful for selecting cells which have incorporated the vector following a transformation procedure, and further comprises the T7 promoter and T7 terminator sequences in operable linkage to a sDR6 coding region. Plasmid pET11A (obtained from Novogen, Madison Wis.) is a suitable parent plasmid. pET11A is linearized by restriction with endonucleases NdeI and BamHI. Linearized pET11A is ligated to a DNA fragment bearing NdeI and BamHI sticky ends and comprising the coding region of the sDR6 gene as disclosed by SEQ ID NO:1 or a fragment thereof.

The sDR6 DNA used in this construction may be slightly modified at the 5′ end (amino terminus of encoded protein) in order to simplify purification of the encoded protein product. For this purpose, an oligonucleotide encoding 8 histidine residues is inserted after the ATG start codon. Placement of the histidine residues at the amino terminus of the encoded protein serves to enable the IMAC one-step protein purification procedure.

### EXAMPLE 7

### Prokaryotic Expression and Purification of sDR6 Protein

An expression vector that carries an open reading frame (ORF) encoding sDR6 or fragment thereof and which ORF is operably-linked to an expression promoter is transformed into E. coli BL21 (DE3)(hsdS gal •cIts857 ind1Sam7nin5lacUV5-T7gene 1) using standard methods. Transformants, selected for resistance to ampicillin, are chosen at random and tested for the presence of the vector by agarose gel electrophoresis using quick plasmid preparations. Colonies which contain the vector are grown in L broth and the protein product encoded by the vector-borne ORF is purified by immobilized metal ion affinity chromatography (INAC), essentially as described in U.S. Pat. No. 4,569,794.

Briefly, the IMAC column is prepared as follows. A metal-free chelating resin (e.g., Sepharose 6B IDA, Pharmacia) is washed in distilled water to remove preservative substances and infused with a suitable metal ion [e.g., Ni(II), Co(II), or Cu(II)] by adding a 50 mM metal chloride or metal sulfate aqueous solution until about 75% of the interstitial spaces of the resin are saturated with colored metal ion. The column is then ready to receive a crude cellular extract containing the recombinant protein product.

After removing unbound proteins and other materials by washing the column with any suitable buffer, pH 7.5, the bound protein is eluted in any suitable buffer at pH 4.3, or preferably with an imidizole-containing buffer at pH 7.5.

### EXAMPLE 8

### Production of an Antibody to sDR6

Substantially pure sDR6 or fragments thereof is isolated from transfected or transformed cells using any of the methods well known in the art, or by a method specifically disclosed herein. Concentration of protein in a final preparation is adjusted, for example, by filtration through an Amicon filter device such that the level is about 1 to 5 μg/ml. Monoclonal or polyclonal antibody can be prepared as follows.

Monoclonal antibody can be prepared from murine hybridomas according to the method of Kohler and Milstein (see, Kohler and Milstein, Nature, 256:495 (1975)), or a modified method thereof. Briefly, a mouse is repetitively inoculated with a few micrograms of the protein or fragment thereof, or fusion peptide thereof, over a period of a few weeks. The mouse is then sacrificed and the antibody producing cells of the spleen isolated. The spleen cells are fused by means of polyethylene glycol with mouse myeloma cells. Fused cells that produce antibody are identified by any suitable immunoassay, for example, ELISA, as described in E. Engvall, Meth. Enzymol., 70, 419, 1980.

Polyclonal antiserum can be prepared by well known methods (See, e.g., J. Vaitukaitis et. al., Clin. Endocirnol. Metab. 33:988 (1971)) that involve immunizing suitable animals with the proteins, fragments thereof, or fusion proteins thereof, disclosed herein. Small doses (e.g., nanogram amounts) of antigen administered at multiple intradermal sites appears to be the most reliable method.

### EXAMPLE 9

### Construction of sDR6-Flag Expression Vector

To facilitate confirmation of DR6 expression (without the use of antibodies), a bicistronic expression vector (pIG1-sDR6F) is constructed by insertion of an “internal ribosome entry site”/enhanced green fluorescent protein (IRES/eGFP) PCR fragment into the mammalian expression vector pGTD (Gerlitz, B. et al. Biochemical Journal 295:131 (1993)). This new vector, designated pIG1, contains the following sequence landmarks: the E1a-responsive GBMT promoter (D. T. Berg et al. BioTechniques 14:972 (1993); D. T. Berg et al. Nucleic Acids Research 20:5485 (1992)); a unique BclI cDNA cloning site; the IRES sequence from encephalomyocarditis virus (EMCV); the eGFP (Clontech) coding sequence (Cormack, et al., Gene 173:33 (1996); the SV40 small “t” antigen splice site/poly-adenylation sequences; the SV40 early promoter and origin of replication; the murine dihydrofolate reductase (dhfr) coding sequence; and the pBR322 ampicillin resistance marker/origin of replication.

A pair of primers containing the DNA sequence cleaved by the restriction enzyme BclI at their 5′ termini are synthesized so that when used to amplify the sDR6 DNA they incorporate the DNA sequence encoding the eight amino acid Flag epitope (nucleotides 24-47 of SEQ ID NO:4) (Micele, R. M. et al. J. Immunol. Methods 167:279 (1994) in-frame with the DNA sequences encoding DR6 at the 3′ terminus of the amplified product. These primers are used to PCR amplify the sDR6 DNA. The resultant PCR product (sDR6F) is then digested with BclI (restriction sites incorporated into the primers) and ligated into the unique BclI site of pIG1 to generate the plasmid pIG1-sDR6F. The human DR6 cDNA orientation and nucleotide sequence is confirmed by restriction digest and double stranded sequencing of the insert.

### EXAMPLE 10

### Construction of sDR6 Non-Flag Expression Vector

In order to generate a non-Flagged expression vector (pIG1-DR6), the 24-base DNA sequence encoding the eight amino acid FLAG epitope is deleted from the pIG1-DR6 construct using the Quik Change mutagenesis kit (Stratagene). A 35-base primer, and its complement, with identity to the 19-base sequences flanking the FLAG sequence is synthesized and used to prime PCR using the plasmid as template. The PCR product is digested with DpnI restriction endonuclease to eliminate the parental DNA, and the digested product is transformed into Epicurean XLI-Blue E. coli cells. Ampicillin-resistant transformants are picked and the plasmid DNA is analyzed by restriction digestion. Precise deletion of the 24-base sequence is confirmed by DNA sequencing of pIG1-sDR6.

### EXAMPLE 11

### Construction of sDR6 Immunoglobulin Fusion Proteins

A. Preparation of sDR6-Fc Fusion Proteins

The extracellular portion of DR6 is prepared as a fusion protein coupled to an immunoglobulin constant region. The immunoglobulin constant region may contain genetic modifications including those which reduce or eliminate effector activity inherent in the immunoglobulin structure. (see, e.g., PCT Publication No. WO88/07089, published Sep. 22, 1988). Briefly, PCR overlap extension is applied to join DNA encoding the extracellular portion of DR6 to DNA encoding the hinge, CH2 and CH3 regions of human IgG1. This is accomplished as described in the following subsections.

B. Preparation of Gene Fusions

A DNA fragment corresponding to the DNA sequences encoding a portion of the sDR6 was prepared by polymerase chain reaction (PCR) using primer pairs designed so as to amplify sequences encoding the DR6 extracellular domain, leader sequence, and including a small amount of 5′noncoding sequence of SEQ ID NO:4. A cDNA encoding full-length DR6 served as the template for amplifying the ECD. PCR amplification generated a DNA fragment which encoded amino acid residues 1-200 of SEQ ID NO:3.

In a second PCR reaction, a second set of primers was designed to amplify the IgG constant region (i.e., the hinge, CH2, and CH3, domains) such that the reverse primer had a unique restriction site and the sequence of the forward primer had a 5′ terminus that is complementary to the 5′ terminal region of the reverse primer used in the sDR6 amplification and would enable the open reading frame in the DR6 extra cellular domain encoding nucleotide sequence to continue throughout the length of the IgG nucleotide sequence. The sequence of human IgG1 was obtained through Genbank (accession no. HUMIGCC4; Takahashi et. al, Cell 29:671-679 (1982)). The target DNA in this reaction was the human genomic DNA encoding IgG heavy chain (Ellison et al., 1982, Nuc. Acids. Res. 10:4071-4079) and was amplified using Human Lymph Node QUICK-Clone™ cDNA purchased from Clontech (cat# 7164-1) as template.

PCR reactions were prepared in 100 μl final volume composed of Pfu polymerase and buffer (Stratagene) containing primers (1 μM each), dNTPs (200 μM each), and 1 ng of template DNA.

The complete sDR6-Fc fusion segment was prepared by performing another PCR reaction. The purified products of the previous two PCR reactions above were mixed, denatured (95° C., 1 minute) and then renatured (54° C., 30 seconds) to allow complementary ends of the two fragments to anneal. The strands were filled in using dNTPs and Taq polymerase and the entire fragment was amplified using the forward PCR primer of the first PCR reaction and the reverse PCR primer of the second PCR reaction. For convenience of cloning into the expression vector, the resulting fragment was then cleaved with restriction enzymes which recognize unique sites incorporated into the forward PCR primer of the first PCR reaction and the reverse PCR primer of the second PCR reaction. The digested fragment was then cloned into an expression vector, pIG1, that had been similarly restricted.

The pIG1 cloning resulted in a clone, pLGD703, that contained the sDR6-Fc fusion. However, sequencing of this clone revealed deletions within the primer sequence region. To correct these errors, two new primers were synthesized, Lars798 (5′- gccgagatctttcgaagccaccatgatcgcgggctccctt -3′) and Lars799, (5′- gtgccgagatctttcgaagccaccatgatcgcgggctcccttctcctg -3′) were used to amplify the sDR6-Fc sequence from pLGD703. PCR reactions as described above were performed with 1 ng of pLGD703 and 0.2 μM of the primers. These reactions were combined and then digested with the restriction enzymes Bgl II and BamHI. The DNA was gel purified and ligated to pIG1 that was cut with BclI and treated with calf intestinal alkaline phosphatase (CIAP). The ligation was used to transform DH5a and the plasmid pLGD715 was identified. The insert was sequenced and it was shown to be correct for the deletions in the primer region.

C. Isolation of Stable Clones

Two cell lines were transformed with pLGD715. First, 293T cells were grown and a transient transfection utilizing lipofectamine (GIBCO-BRL) was performed. Characterization of the supernatant revealed a protein of the size one would expect for a dimer of the sDR6-Rc, thereby confirming the integrity of the pLGD715 construct. The expression of the protein was confirmed by a Western blot utilizing an antibody to human IgG1.

To produce cell lines stably expressing the sDR6-Fc fusion protein, the Syrian hamster cell line AV12-RGT18 was transfected with pLGD715 by the calcium chloride precipitation method (Promega). Two days after the transfection the cells were washed and then trypsinized. The cells were collected and resuspended in 10 ml of the appropriate media. The transfected cells were plated onto gridded Falcon 3025 plates at 1/10, 1/50, and 1/250 in a final volume of 35 ml. The media contained methotrexate at 250 nm concentration. pIG1 contains a copy of the DHFR gene and when amplified will convey methotrexate resistance on the transfected cells. After two to five days, colonies were identified in the 1/50 and 1/250 dilution platings, transferred to microtiter plates, and grown under selection. The ability of these clones to produce the sDR6-Fc protein was examined in serum free media. Many clones were identified that produced the desired protein, one of which was isolated and grown up in 80 roller bottles. The media was collected and the sDR6-Fc fusion protein was isolated as described below.

Those skilled in the art are aware of various considerations which influence the choice of expression vector into which the sDR6-IgG fusion segment can be cloned, such as the identity of the host organism and the presence of elements necessary for achieving desired transcriptional and translational control. For example, if transient expression is desired, the sDR6-IgG fusion segment generated supra can be cloned into the expression vector pcDNA-1 (Invitrogen). Alternatively, stable expression of the fusion protein can be achieved by cloning the DR6-IgG fusion segment into the expression vector pcDNA-3 (Invitrogen).

Alternatively, sDR6-IgG fusion proteins can be generated using an expression vector such as the CD5-IgG1 vector (described by Aruffo et al., Cell, 61:1303-1313 (1990)), which already contains the IgG constant region. According to this method, the DNA fragment encoding the DR6 extracellular domain is generated in a PCR reaction so that the open reading frame encoding the DR6 extracellular domain is continuous and in frame with that encoding the IgG constant region.

For example, the extracellular domain (including signal peptides) of DR6 is PCR amplified. Each forward primer above contains an appropriate restriction site and each reverse primer above contains an appropriate restriction site. After amplification using a DR6 cDNA as a template, the resulting PCR fragment containing the sDR6 encoding cDNA is cloned into the CD5-IgG vector (Aruffo et al., (1990), Cell). The resulting vectors are transiently transfected into COS cells and conditioned media is generated. Immunoprecipitation (IP) of the conditioned media with protein A and analysis by SDS PAGE reveals whether the desired protein is expressed. To improve expression of the human sDR6-IgG fusion, primers are designed which amplify the sDR6-IgG fusion (without the signal peptide) and this fragment is co-ligated with sequences encoding other signal-peptides such as that from the mouse DR6 into the CD5-IgG vector.

After amplification, restriction enzyme digestion, and subcloning, the resulting construct is transiently expressed in COS cells. IP and SDS-PAGE analysis of the resulting conditioned media shows whether expression of the human sDR6 IgG fusion is successful. An alternative method for enhancing the expression of immunoglobulin fusion proteins involves insertion of the DR6 extracellular domain (not including the signal peptide) into the CD5-IgG1 vector in such a manner so that the CD5 signal peptide is fused to the mature DR6 extracellular domain. Such a signal peptide fusion has been shown to improve expression of immunoglobulin fusion proteins.

D. Preparation of Modified CH2 Domains

The nucleotide sequence of the sDR6-IgG gene fusions described supra can be modified to replace cysteine residues in the hinge region with serine residues and/or amino acids within the CH2 domain which are believed to be required for IgG binding to Fc receptors and complement activation.

Modification of the CH2 domain to replace amino acids thought to be involved in binding to Fc receptor is accomplished as follows. The plasmid construct generated supra provides the template for modifications of the sDR6-IgCγ1 CH2 domain. The template can be PCR amplified using the forward PCR primer described in the first PCR reaction supra and a reverse primer designed such that it is homologous to the 5′ terminal portion of the CH2 domain of IgG1 except for five nucleotide substitutions designed to change amino acids 234, 235, and 237 (Canfield, S. M. and Morrison, S. L., J. Exp. Med., 173:1483-1491 (1991)) from Leu to Ala, Leu to Glu, and Gly to Ala, respectively. Amplification with these PCR primers will yield a DNA fragment consisting of a modified portion of the CH2 domain. In a second PCR reaction, the template can be PCR amplified with the reverse primer used in the second PCR reaction supra, and a forward primer; the forward primer is designed such that it is complementary to the Ig portion of the molecule and contains the five complementary nucleotide changes necessary for the CH2 amino acid replacements. PCR amplification with these primers yields a will provide a cDNA fragment consisting of the modified portion of the CH2 domain, an intron, the CH3 domain, and 3′ additional sequences. The complete sDR6-IgCγ1 segment consisting of a modified CH2 domain is prepared by an additional PCR reaction. The purified products of the two PCR reactions above are mixed, denatured (95° C., 1 minute) and then renatured (54° C., 30 seconds) to allow complementary ends of the two fragments to anneal. The strands are filled in using dNTP and Taq polymerase and the entire fragment amplified using forward PCR primer of the first PCR reaction and the reverse PCR primer of the second PCR reaction. For convenience of cloning into the expression vector, the resulting fragment is then cleaved with restriction enzymes recognizing sites specific to the forward PCR primer of the first PCR reaction and the reverse PCR primer of the second PCR raction. This digested fragment is then cloned into an expression vector that has also been treated with these restriction enzymes.

Sequence analysis is used to confirm structure and the construct is used to transfect COS cells to test transient expression. hIgG ELISA is used to measure/confirm transient expression levels approximately equal to 100 ng protein/ml cell supernatant for the construct. CHO cell lines are transfected for permanent expression of the fusion proteins.

### EXAMPLE 12

### Isolation of a High-Producing TNFR6-Fc AV12 Cell Line from AV12 Transfectants

The recombinant plasmid carrying the sDR6-Fc cDNA insert also encodes resistance to methotrexate. In addition, the construct contains a gene encoding a fluorescent protein, GFP, on the same transcript and immediately 3′ to the sDR6-Fc cDNA insert. Since high level expression of GFP would require a high level of expression of the sDR6-Fc mRNA, highly fluorescent clones would have a greater probability of producing high levels of sDR6-Fc. pIG1-sDR6-Fc are used to transfect AV12 cells. Cells resistant to 250 nM methotrexate are selected and pooled. The pool of resistant clones is subjected to fluorescence assisted cell sorting (FACS), and cells having fluorescence values in the top 5% of the population are sorted into a pool and as single cells. The high fluorescence pools are subjected to three successive sorting cycles. Pools and individual clones from the second and third cycles are analyzed for sDR6 production by SDS-PAGE. Pools or clones expressing the DR6 proteins at the highest level judged from Coomassie staining or Western blotting are used for scale-up and purification of the expressed protein.

### EXAMPLE 13

### Purification of a sDR6 Immunoglobulin Fusion Protein from Media of Transfected AV12 Cells

AV12 cells transformed with a vector containing a cDNA insert encoding a DR6-IgG fusion protein are grown in culture bottles until confluent. Media is collected, concentrated approximately 20 fold, and clarified by centrifugation. The media concentrate is pumped onto a Ni loaded iminodiacetic acid column. The column is washed with 100 mM sodium phosphate, 100 mM sodium chloride buffer (pH 7.5). Bound protein is eluted with a pH gradient from pH 7.5 to 4.25.

Fractions containing the sDR6-IgG fusion protein are pooled, diluted 1:1 with 50 nM sodium phosphate (pH 5.6), and pumped onto a cation exchange column (TSK-SP 5PW). The column is washed with 50 mM sodium phosphate (pH 5.6) and bound protein eluted with a gradient from 0 to 0.5 M sodium chloride. Fractions containing sDR6-IgG fusion are pooled and dialyzed into phosphate buffered saline (pH 7.5).

The identity of the protein is confirmed by digesting the protein with trypsin and analyzing the resulting peptides by mass spectroscopy and tandem MS/MS analysis.

### EXAMPLE 14

### Large-Scale Protein Purification of sDR6 from Stable Cell Line

Large-scale production of sDR6 is done by first growing stable clones in several 10 liter spinners. After reaching confluency, cells are further incubated for 2-3 more days to secrete maximum amount of sDR6 into the media. Media containing sDR6 is adjusted to 0.1% CHAPS and concentrated in an Amicon ProFlux M12 tangential filtration system to 350 ml. The concentrated media is centrifuged at 19,000 rpm (43,000×g) for 15 minutes and passed over a SP-5PW TSK-GEL column (21.5 mm×15 cm; TosoHaas) at a flow rate of 8 ml/min. The column is washed with buffer A(20 mM MOPS, 0.1% CHAPS, pH 6.5) until the absorbency (280 nm) returns to baseline and the bound proteins are eluted with a linear gradient from 0.1 M-0.3 M NaCl (in buffer A) developed over 85 min. Fractions containing sDR6 polypeptides are pooled and passed over a (7.5 mm×7.5 cm) Heparin-5PW TSK-GEL column equilibrated in buffer B (50 mM Tris, 0.1% CHAPS, 0.3M NaCl, pH 7.0). The bound protein is eluted with a linear gradient from 0.3M-1M NaCl (in buffer B) developed over 60 min. Fractions containing sDR6 polypeptides are pooled and passed over a 1 cm×15 cm Vydac C4 column equilibrated with 0.1% TFA/H20. The bound sDR6 is eluted with a linear gradient from 0-100% CH3CN/0.1% TFA. Fractions containing sDR6 are analyzed by SDS-PAGE and found to be greater than 95% pure and are dialyzed against 8 mM NaPO4, 0.5 M NaCl, 10% glycerol, pH 7.4. The N-terminal sequence of the sDR6 polypeptide can be confirmed using the purified protein.

### EXAMPLE 15

### Purification of sDR6 Polypeptides

More generally, a sDR6 can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, size exclusion chromatography, and lectin chromatography. Preferably, high performance liquid chromatography (“HPLC”), cation exchange chromatography, affinity chromatography, or size exclusion chromatography, or combinations thereof, are employed for purification. Particular methods of using protein A or protein G chromatography for purification are known in the art. Both protein A and protein G binds the Fc regions of IgG antibodies, and therefore makes a convenient tool for the purification of sDR6 molecules containing the IgG region. sDR6 purification is meant to include purified parts of the chimeric (the extracellular region and the immunoglobulin constant region) that are purified separately and then combined by disulfide bonding, x-linking, and the like.

### EXAMPLE 16

### Cell Binding Assays for sDR6

Cell binding assays with a sDR6 can be performed using cells that present DR6 ligands on their cell surface. Cells are washed and incubated for 30 minutes at 4° C. in Hanks Balance Salt Solution (HBSS) (supplemented with 10% bovine calf serum and 0.1% NaN₃) containing sDR6:Fc at 5 μg/ml, washed, and then stained with goat anti-human IgG conjugated with phycoerythrin (anti-huIg-PE). Stained cells can be analyzed by flow cytometry (FACSCaliber, Becton-Dickinson). Binding of the sDR6:Fc can be determined by incubating various concentrations of sDR6:Fc or control IgG with the ligand presenting cells. sDR6 binding can be determined by calculating the fluorescence intensity=(mean fluorescent channel)(% positive fluorescent events), where a positive event has a fluorescence value>98% of the value for normal IgG. Specific fluorescence intensity can be represented by the fluorescence after subtraction of the value for control IgG.

### EXAMPLE 17

### Immunoprecipitation Binding Assay for sDR6 Polypeptides

Essentially, 2 μg of purified sDR6-Fc is incubated with 250 ng of various purified soluble ligands in 250 μl of 25 mM HEPES, pH 7.2, 0.25% bovine serum albumin, 0.01% Tween in RPMI 1640 at 4° C. for 2 hours. Protein A-Sepharose 4B (Amersham, 30 μl of a 75% slurry) is added and incubated for an additional 1 hour. Complexes are recovered by centrifugation, washed three times with binding buffer, and electrophoresed on a 15% polyacrylamide gel, and then transferred to nitrocellulose for Western blot analysis with Protein A capture and detection of bound ligand or anti-His repeat (CLONTECH) monoclonal antibodies, or, as the case may be, using an epitope-tag.

### EXAMPLE 18

### Proliferation Assay

Biological effects of a sDR6 polypeptide can be measured in a T cell proliferation assay. Essentialy purified T cells from the spleen or lymph nodes (4×105) in 200 μl RPMI and 10% FBS media are seeded in triplicate in 96 well plates that are coated with different concencentrations of anti-CD3 or combined with anti-CD3 and anti-CD28. Cells are pulsed for 12 hours with 1 μCi of [3H] thymidine in the presence and/or absence of varying concentrations of sDR6. Thymidine incorporation is quantified using a scintiallation counter.

### EXAMPLE 19

### Generation of DR6 Deficient Mice

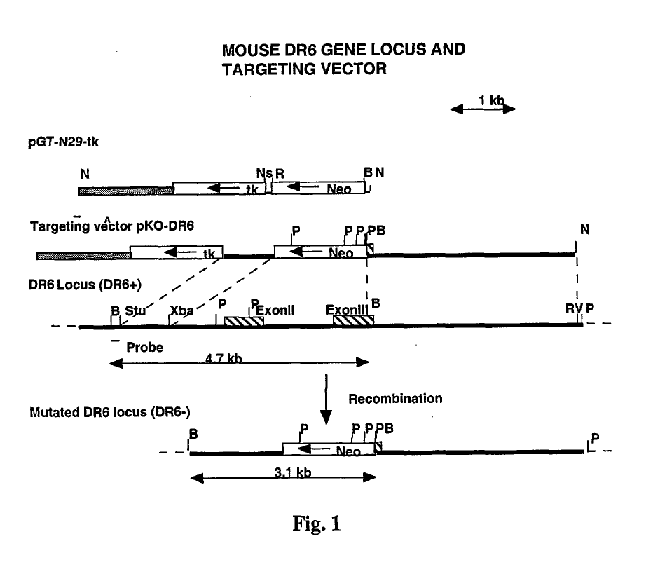

Genomic DNA clones corresponding to the DR6 locus were cloned from a FixII phage library prepared from mouse strain 129/Sv (Stratagene). A targeting vector (pKO-DR6) was constructed in the vector pGT-N29 (New England BioLabs) by replacing a 3.4 kb XbaI-BamHI genomic fragment of DR6 encompassing the translation initiation site with a neomycin resistance cassette (pGK-neo) (FIG. 1). More specifically the targeting vector (pKO-DR6) contained a 1.2-kb StuI-XbaI fragment obtained from the 5′ end of the DR6 genomic clone inserted into the vector at the NsiI and EcoRI sites and a 3.5-kb BamHI-EcoRV fragment derived from the 3° end of the DR6 genomic clone inserted into the vector at the BamHII and NotI sites, using appropriate linkers (FIG. 1). The neomycin resistance cassette was placed in the anti-sense orientation to DR6 transcription.

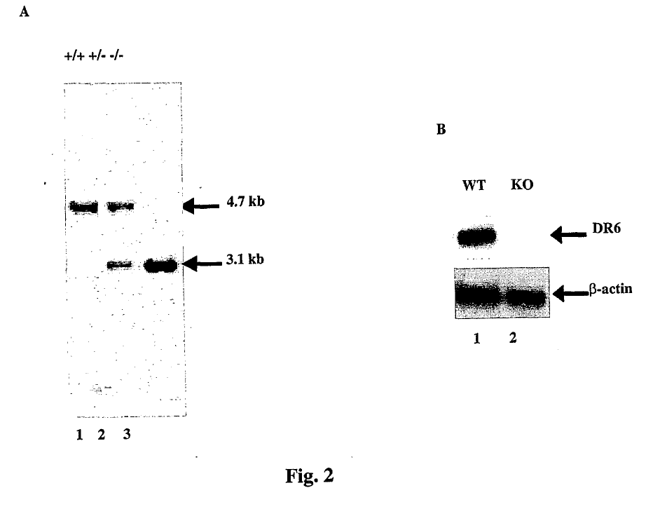

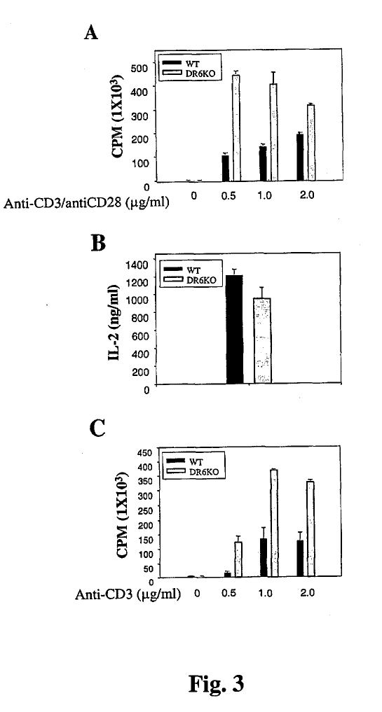

R1 ES cells were electroporated with pKO-DR6 previously linearized with NotI. Genomic DNA from 146 transfectants resistant to G418 (300 μg/ml; GIBCO/BRL) was treated with BamHI and characterized by Southern blot analysis using probe A (FIG. 1). The probe was a 200 bp BamHI-StuI fragment of the DR6 locus. Thirteen ES clones with a hybridization pattern consistent with the desired homologous recombination were identified. Two targeted ES clones were injected into 3.5-day-old C57BL/6(B6) blastocysts resulting in chimeric mice that transmitted the disrupted DR6 allele through the germ line. The contribution of embryonic stem cells to the germline of chimeric mice was assessed by breeding with B6 mice. Germline transmission of the DR6 mutation was confirmed by Southern analysis of tail DNA. Specifically, the presence of 3.1- and 4.7-kb hybridizing bands were indicative of mutated and wild type alleles, respectively (FIG. 2A). Mice heterozygous for the mutant gene were interbred to homozygosity (FIG. 2A). The null mutation of DR6 was demonstrated by the absence of DR6 expression, as determined by Northern analysis of mRNA isolated from kidneys of wild type (WT) and homozygous mutant mice (FIG. 2B).

Kidneys from wild type and DR6 knockout (DR6 KO) mice were used for total RNA preparation with the TRIzol reagent protocol as recommended by the manufacturer. Poly-A⁺ RNA was prepared from the total RNA using Oligotex (Qiagen). Northern Blot analysis was performed with poly A⁺ RNA from wild type and DR6 deficient mouse kidneys. Homozygous DR6 deficient mice were born at the expected Mendelian ratio, fertile and showed no apparent phenotypic abnormalities.

### EXAMPLE 20

### Stimulation of T-cell Growth in a CD28 Independent Manner in DR6 Deficient T cells

Purified T cells from spleen or lymph nodes (4×10⁵) in 200 μl RPMI and 10% FBS media were seeded in triplicate in 96 well plates that had been coated with different concentrations of anti-CD3 or combined anti-CD3 and anti-CD28 (Pharmingen). Cells were pulsed for 12 hr with 1 μCi of [³H] thymidine, and [³H] thymidine incorporation was quantitated using a scintillation counter. T cell activation-induced cell death assays were performed with splenocytes from wild type and DR6−/−mice. Splenocytes were cultured with plate-bound anti-CD3 (10 μg/ml) for 24 hr, followed by culture in media containing RPMI, 10% FBS and IL-2 (30 ng/ml) for 4 days. The activated lymphocytes were treated with plate-bound anti-CD3 (5 μg/ml) for 24 hrs. followed by apoptosis assays with Annexin V (Pharmingen) staining and flow cytometry.

Purified T cells from spleen were cultured in microtiter plates coated with anti-CD3 at suboptimal doses and anti-CD28 mAbs. The ³H-thymidine incorporation was determined after a 48-72 hour incubation. The proliferation of T cells from DR6 deficient mice was enhanced 2-4 fold in response to anti-TCR/anti-CD28 stimulation compared with wild type T cells (FIG. 3A). The level of IL-2 secreted into the supernatants of these primary cultures showed no significant differences (FIG. 3B). DR6 appearred to negatively regulate T cell growth through the engagement of T cell receptor, as the absence of DR6 resulted in enhanced T cell proliferation of mutant T lymphocytes. Moreover, the observed enhanced proliferation was intrinsic to the mutant cell and was not due to the higher IL-2 production.

Purified T cells isolated from spleens of DR6 deficient mice and wild-type littermates were also stimulated only in the presence of anti-CD3. T cells from DR6 deficient mice showed about a 4-fold enhanced proliferation compared with wild-type T cells (FIG. 3C). Therefore, DR6 suppresses T cell growth in a CD28-independent manner.

Activation induced cell death (AICD) assays were also performed with wild type and DR6 deficient mice (data not shown). These studies indicated that DR6 acts as a negative regulator in mediating peripheral T-cell proliferation through a pathway distinct from AICD (i.e., the enhanced proliferation was not due to higher IL-2 production).

### EXAMPLE 21

### Hyperproliferation of DR6 Deficient CD4⁺ T Cells

Lymph node cells from wild type and DR6 deficient mice were washed 3× with RPMI media plus 10% Fetal Calf serum. Cells were incubated with anti-CD8 or anti-CD4 and anti-I-A^{b} (Pharmingen) for 30 minutes. The cell and the antibody mixture was then incubated with goat anti-rat IgG (H & L) labeled magnetic beads (PerSeptive Biosystems) for 20 min. followed by magnetic field separation to remove MHC class II, CD8- or CD4-expressing cells. Cell purity was determined using FITC-labeled anti-CD4 antibody (Pharmingen) followed by the Flow Cytometry analysis. This procedure generates 90-96% CD4⁺ cells. Alternatively, labeled CD4⁺ and CD8⁺ cells were sorted by a fluorescence cell sorter. Purified CD4⁺ and CD8⁺ T cell populations cultured in 96-well plates coated with anti-CD3 and anti-CD28 demonstrated that DR6-deficiency has a profound effect on the proliferative responses of the CD4⁺ T-cell subset (FIG. 4A).

### EXAMPLE 22

### Increased Th2 Cell Differentiation in DR6-deficient Mice

When activated by APCs, naïve CD4⁺ T cells undergo clonal proliferation and produce IL-2. The activated CD4⁺ T cells may then become Th1 or Th2 effector cells, which mediate inflammatory or humoral responses, respectively. Although the polarization of Th cell differentiation is, at least in part, determined by the cytokine environment, signals from the TCR-CD3 complex and from the costimulatory factor CD28 may also affect cytokine production by mechanisms not yet understood (Constant and Bottomly, 1997). To investigate the effects of DR6 deficiency on Th cell differentiation, cytokine production by purified CD4⁺ T cells after stimulation with anti-CD3 and anti-CD28 was measured.

Quantitation of cytokines in cell culture supernatants after a variety of in vitro stimulations was performed by a sandwich ELISA as described (Homann et al., 1999). Briefly, 96-well plates were precoated with 2 μg/ml capture antibodies (IL2, IL-4, IL-5, IL-6, IL-10, IFN-γ) (R&D systems) overnight at 4° C. After washing with PBS/Tween 20 and blocking with blocking buffer (PBS, pH7.4, 1% Fetal Calf Serum), serial dilution of recombinant cytokine standards (R&D systems) and sample supernatants were added and incubated for 2 hr at room temperature. Plates were washed 3× with PBS/Tween 20 followed by incubation with 1 μg/ml matched biotinylated detection antibodies (R & D systems) for 2 hr at room temperature. For the color reaction, H₂O₂ activated ABTS (2,2′-Azino-di-[3-ethylbenzthiazolin-6-sulfonic acid]) (Pierce) was added followed by incubation for 30 minutes at room temperature. Plates were read at 405 nm with V max microplate reader (Molecular Devices). Th1 and Th2 cell differentiation was performed according to the method of McKenzie et al (1998). Briefly, purified CD4⁺ cells were cultured on anti-CD3 and anti-CD 28 coated plates (1 μg/ml, BD Pharmingen) in the presence of exogenous cytokines or anti-cytokine antibodies as indicated. IL-2 (10 ng/ml; R&D) was added to all cultures. For the Th1 differentiation assay, IL-12 (5 ng/ml) and anti-IL-4 antibody (2 μg/ml, R&D) were added to the culture, whereas IL-4 (100 ng/ml) and anti-IFN-γ (2 μg/ml, R&D) were used to promote Th2 differentiation.

Disruption of DR6 resulted in augmented cytokine production characteristic of differentiated Th2 cells. Compared with their wild type T cells, DR6-deficient CD4⁺ T cells secreted a higher level of Th2 cytokines such as IL4, IL-5 and IL-10, while the secretion of Th1 cytokines IFN-γ remained comparable to the control CD4⁺ T cells (data not shown). Furthermore, purified CD4⁺ T cells from DR6 deficient mice stimulated with anti-CD3 in the presence of IL-4 and anti-IFN-γ (which normally induces Th2 differentiation) showed drastically increased IL-4 production compared to wild-type CD4⁺ T cells under the same conditions (data not shown). In contrast, when DR6 deficient CD4⁺ T cells were stimulated with anti-CD3 in the presence of IL-12 and anti-IL-4 (which normally induces Th1 responses), there were no obvious differences in the production of the Th1 cytokine IFN-γ between WT and DR6 deficient mice. DR6 deficient CD4⁺ T cells produced 300-fold higher IL-4 compared to wild type T cells under Th1 condition (data not shown).

### EXAMPLE 23

### Enhanced T-cell Priming and Cytokine Production in DR6 Deficient Mice

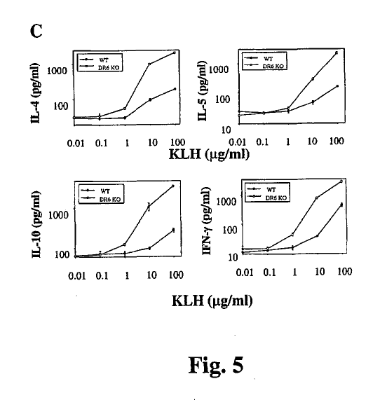

Six-week-old wild-type (WT) and DR6−/−(DR6 KO) female mice (four mice in each group) were immunized with 100 μg keyhole limpet hemocyanin (KLH) or hen egg lysozyme (HEL) in complete Freunds adjuvant (CFA) in the hind footpads. CD4⁺ T cells were purified from the draining lymph nodes and cultured in the presence of antigen presenting cells (APC) and different concentrations of KLH or HEL. APCs used were isolated from the spleens of 6-week-old WT and DR6−/−mice and irradiated (3,000 rads). T cell recall responses were thereby tested. Cytokine secretion by CD4⁺ T cells from KLH immunized mice were assayed by ELISA. A significantly enhanced T-cell proliferative response was observed in DR6 deficient mice, and the antigen dose-response curve was shifted by several orders of magnitude (FIG. 5A). Similarly enhanced responses were seen with another protein antigen, hen egg lysozyme (HEL) (FIG. 5B). These data suggested that DR6 plays an important role in antigen-specific T cell•priming in vivo. To determine whether the enhanced responses in DR6-deficient mice occurred at the level of the APC or intrinsic hyperproliferative response of CD4⁺ T cells, KLH-primed CD4⁺ T cells purified from DR6 deficient and wild type mice were examined for their recall response in the presence of wild type or DR6 deficient APC. The T cell recall response for DR6 deficient CD4⁺ T cells remained at the similar level in the presence of WT APC (FIG. 5C), indicating that DR6 is important to balance antigen-specific T cell responses during both the priming and effector phases of T cell activation.

The culture supernatants of T cell recall responses were assayed by ELISA for cytokine production. The production levels of the Th1 cytokine (IFN-γ), and Th2 cytokines (IL-4, IL-5, IL-10) were significantly higher in DR6 deficient CD4⁺ T cells compared to those of control CD4⁺ T cells (data not shown), suggesting that DR6 is critically involved in both the Th1 and Th2 differentiation during recall responses.

### EXAMPLE 24

### Absence of DR6 Results in Increased NF-ATC Nuclear Transcription

To further understand the potential mechanism through which DR6 regulates T cell responses, the effect of DR6 deficiency on NF-ATc and NF-κB, which are key transcription factors involved in TCR-mediated signaling, was examined. Generally, upon T cell activation, NF-ATc is dephosphorylated and translocated into the nucleus to activate gene transcription, whereas NF-κB is translocated into the nucleus after disassociating from phosphorylated IKB (Olsson et al., 1999).

Purified CD4⁺ T cells from wild type and DR6 deficient lymph nodes were stimulated in vitro with plate-bound anti-CD3/anti-CD28 (1 μg/ml) for 3 days and nuclear extracts for Western blot analysis were prepared. Nuclei were isolated according to known procedures. Equal amounts of extracted proteins were separated on a 10-20% gel by polyacrylamide gel electrophoresis, followed by electrotransfer to nitrocellulose membranes and probed with antibodies specific to NF-ATc, NF-κB p50, respectively, followed by detection with horseradish peroxidase-conjugated secondary antibodies and developed by chemiluminescence (Pierce).

Western blot analyses showed, upon stimulation of CD4⁺ T cells with anti-TCR, the level of NF-ATc in the nucleus were dramatically increased in DR6 deficient T cells without any significant change of total NF-ATc level (FIG. 6A). In contrast, the NF-κB pathway was not affected, as demonstrated by nuclear localization of the p50 Rel A protein after stimulation in both DR6 deficient and wild type CD4⁺ T cells (FIG. 6A). Nuclear levels of another member of NFAT family, NF-ATp was also tested in our study, and no significant difference was observed (data not shown).

### EXAMPLE 25

### Reduced Surface Expression of CTLA-4 and Enhanced CD28 Surface Expression in DR6 Deficient T cells after Stimulation

The cell surface expression of CD28 and CTLA-4 was examined by staining T cells from wild type and DR6 deficient mice with PE-labeled anti-CD28 or anti-CTLA-4 monoclonal antibodies before and after stimulation with anti-CD3. The expression level was then measured with flow cytometry analysis. The cell surface level of CTLA-4 was higher in wild type T cells compared to that of DR6 deficient T cells. In contrast, CD28 expression was much higher on the T cell surface of DR6 deficient mice (data not shown ). Thus, DR6-deficiency results in down-regulation of CTLA-4 surface expression and up-regulation of CD28 after T cell stimulation. Taken together enhanced NF-ATc level in the nucleus combined with the different surface expression of CTLA-4 and CD28 contribute to the observed increase in CD4⁺ T cell proliferation and Th2 cytokine secretions in DR6 deficient T cell responses.

### EXAMPLE 26

### Negative Regulation of Th2 Cell Differentiation

Th2 cytokines; especially IL-4 and IL-5, are critical for IgE production and eosinophil growth and differentiation. IgE and eosinophil are two critical factors for eliciting allergic reactions and/or allergic autoimmune diseases such as asthma, atopy, and eczema. Since DR6 is a potential negative regulator in Th2 cell differentiation, a DR6 agonist may be used to reduce Th2 cell differentiation and Th2 cytokine production thereby inhibit, prevent, delay onset and/or the escalation or progression of allergic diseases and/or autoimmune diseases. Assays for investigating effects on allergy and/or allergy associated conditions are known in the art.

### EXAMPLE 27

### Transplantation of DR6 Deficient Donor T Cells Hastens the Onset and Severity of Acute GVHD in a Murine Parent-into-F₁ Model

A murine parent-into-F1 model of acute GVHD was used to study the ability of DR6 knockout T cells to induce GVHD. Six eight-week-old male C57BL/6 (B6) (H-2K^{b}) and male B6×DBA/2 (BDFL) (H-2K^{b+d}) mice were purchased from the Jackson Laboratory (Bar Harbor, Me.). Balb/c mice were purchased from Harlan (Indianapolis, Ind.). Splenocytes were obtained by macerating spleens in IMDM supplemented with 10% FBS, 2 mM glutamine, 100 U/ml Penicillin, and 100 μg/ml streptomycin with a Dounce homogenizer and then passing the cells through a mesh filter (Falcon). Cells were then layered over Histopaque 1119 (Sigma Diagnostics, St. Louis, Mo.) and centrifuged at 1750 rpm for 15 min. with no brake. Cells at the interface were removed and washed three times with cold PBS supplemented with 2% BSA. T cells were then isolated by positive selection with anti-Thy 1.2 conjugated microbeads and a magnetic cell separation system (Miltenyi Biotec, Auburn, Calif.) according to the manufacturer's instructions. A fraction of isolated T cells were analyzed by immunostaining with a FITC conjugated anti-CD3 antibody and flow cytofluorometric analysis on a FACscan machine (Becton Dickinson, San Jose, Calif.). The purity of T cells was typically greater than 94%.

Isolated T cells were washed in Hanks Balanced Saline Solution (HBSS) twice and resuspended at 5×10⁶ cells/ml. Recipient male BDF1 mice were weighed and then sublethally irradiated with 500 rads of a ¹³⁷Cs source at a rate of 107 rads/min. Irradiated mice were injected with 2.5×10⁶ T cells in 0.5 mls of HBSS via the tail vein within one hour of irradiation. Control mice received 2.5×10⁶ T cell depleted (TCD) splenocytes which consisted mainly of B cells. Mice were monitored daily for symptoms of GVHD such as ruffled fur, hunched posture, inactivity, eye lesions and snout swelling and were weighed every 2 days. Some mice were sacrificed at 5 or 7 days post-transplant for analysis of various parameters.

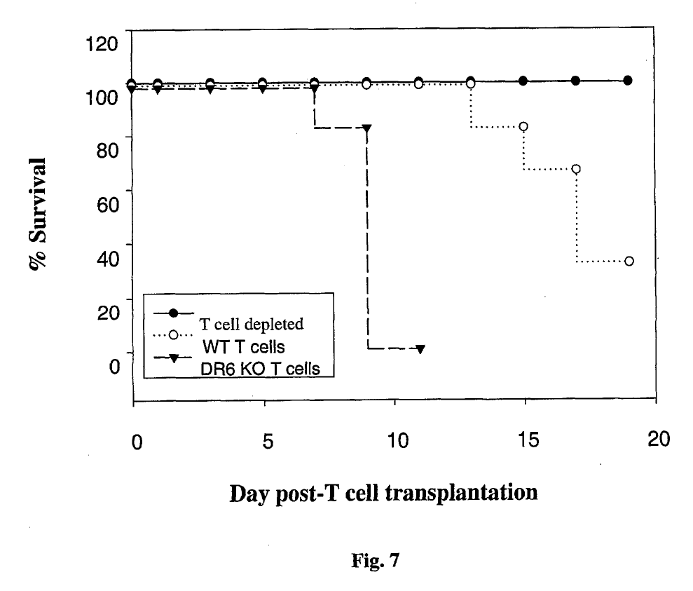

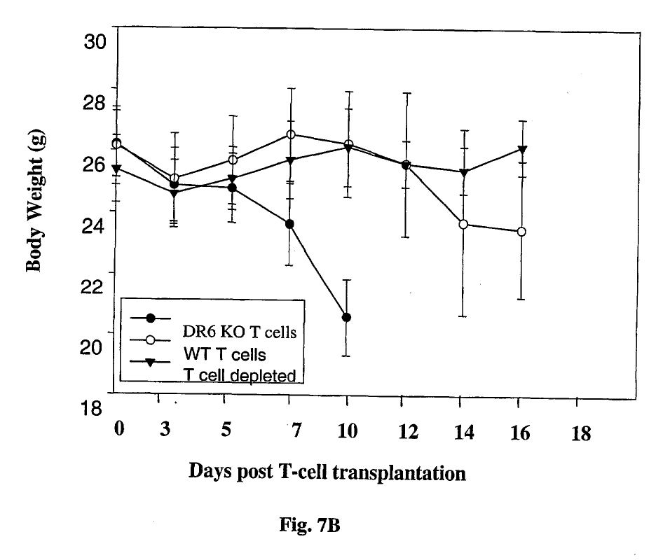

In this model, wild type B6 donor T cells (H-2k^{b}) are engrafted into sublethally irradiated BDF1 recipient mice (H-₂K^{b+d}) and recipients are expected to develop acute GVHD. Typically, GVHD in recipient mice leads to a pronounced weight loss beginning around 14-16 days post-transplant, resulting in 100% mortality by 22-26 days post-transplant (FIG. 7). The weight loss and subsequent death characteristic of GVHD are not observed in T cell depleted (TCD) grafts (FIG. 7). In contrast, BDF1 recipients of DR6 deficient donor T cells (H-2K^{b}) exhibited a much more rapid weight loss than even the wild type T cell recipients that began around 5 days post-transplant (FIG. 7). The most rapid reduction in weight was observed between days 7 and 10. This earlier onset of rapid weight loss resulted in a striking increase in rate of mortality of BDF1 recipients of DR6 deficient donor T cells (H-2K^{b}) which reached 100% by 11 days post-transplant (FIG. 7).

The onset of GVHD, as determined by outward physical symptoms such as hunched posture, ruffled fur, inactivity, eye lesions and swelling of the skin around the snout was apparent in mice receiving DR6 deficient grafts at a much earlier time post-transplant than mice receiving wild type grafts (data not shown). Physical symptoms of GVHD were apparent as early as 5 days post-transplant in DR6 deficient recipients, while these symptoms appeared in wild type recipients at a much later time (around 14-18 days post-transplant).

Various tissues of transplanted mice having been necropsied at 7 days post-transplant were fixed in fresh 4% paraformalde-hyde and transferred into 70% ethanol. The tissues were subsequently processed by dehydration, clearing and embedding in paraffin according to standard histological procedures. Blocks were sectioned at 5 μm and prepared for immunohistochemistry by mounting of sections onto electrostatically charged slides (ProbeOn Plus, Fisher Scientific). Tissue sections were rehydrated, quenched of endogenous peroxidase, and blocked in normal serum. The tissue sections were then incubated with an anti-activated caspase-3 antibody (R&D Systems), followed by incubation with biotinylated secondary antibody and visualized with streptavidin complex (DAKO) with diaminobenzidine as the chromagen. For terminal deoxyribose-UTP nick end labeling (TUNEL), tissue sections were rehydrated, permeablized with proteinase K, and labeled with digoxygenin linked dUTP (Intergen Co.). Incorporated dUTP was located with a biotinylated anti-digoxygenin antibody and visualized with streptavidin complex with diaminobenzidine as the chromagen.

Although other target organs exhibited ongoing GVHD pathology, the severity of GVHD was most pronounced in the spleen and small intestine of mice receiving DR6 deficient grafts. TUNEL assays to detect apoptotic cells, showed prominent cell death in the crypt epithelium of the small intestine, extending along the villous epithelium for both wild type and DR6 deficient grafts (data not shown). However, the frequency of apoptotic cells was visibly higher in mice with DR6 deficient donors compared to wild type donors, while very few apoptotic cells were observed in mice having TCD donors (data not shown). Apoptotic and necrotic intestinal epithelial cells appeared to be sloughed into the lumen predominantly in recipients of DR6 deficient donors. Immunostaining for caspase 3 also revealed a number of cells undergoing apoptosis in recipient spleens (data not shown). Again, there was visibly greater immunostaining for caspase 3 in mice that received DR6 deficient grafts compared to wild type, with undetectable immunostaining for TCD grafts (data not shown). The spleens of DR6 deficient recipients exhibited extensive ongoing apoptosis in the perifollicular red pulp and marginal zone areas (data not shown).

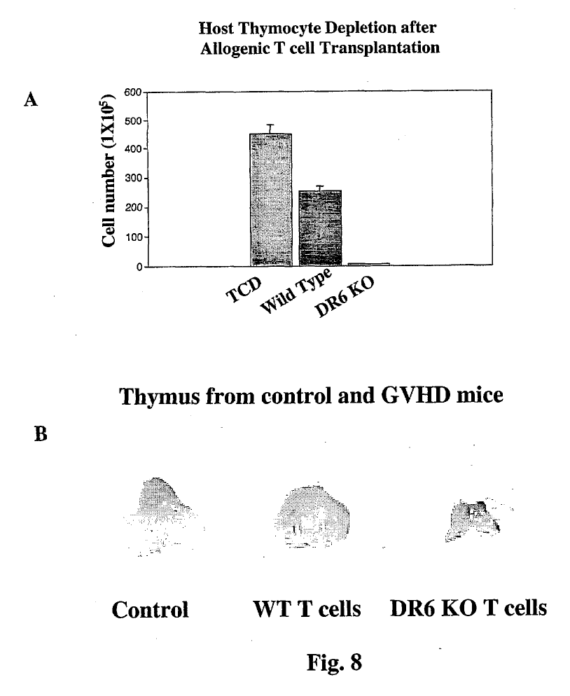

The thymus in mice receiving DR6 deficient grafts was extremely small at 7 days post-transplant (FIG. 8B). While TCD recipient mice contained, on average, around 4-5×10⁷ thymocytes, recipients of wild type grafts were about 2-fold lower, indicating some loss in thymic cellularity (FIG. 8A). Strikingly, recipients of DR6 deficient grafts contained only a very small number of thymocytes (approx. 5×10⁵) indicating significant thymic GVHD (FIG. 8A). As expected, the analysis of thymocytes by flow cytometry revealed a small reduction in CD4⁺CD8⁺ T cells for recipients of wild type grafts (77% vs. 88%; data not shown). However, the number of thymic CD4⁺CD8⁺ T cells in recipients of DR6 deficient grafts was much lower compared to that observed with wild type grafts (12% vs. 77%; data not shown).

Antibodies specific to the mouse MHC marker H-2K^{d} were used to distinguish host cells from donor cells. Donor cells were identified in histograms by the absence of H-2K^{d} staining. Flow cytometric analysis was carried out by resuspending cells in PBS/1% BSA at 1×10⁶ cells/ml in a volume of 200 μl/sample. FITC-conjugated anti-mouse H-2K^{d}, PE-conjugated anti-mouse CD4, CD8 and CD19 were used for staining. CD4⁺CD8⁺ T cells in the thymus were also analyzed with a FITC-conjugated anti-mouse CD4 antibody and a PE-conjugated anti-mouse CD8 antibody. Activation of donor T cells by alloantigens in vivo was assessed with FITC-conjugated anti-mouse CD25, PE-conjugated anti-mouse CD28, and PE-conjugated anti-mouse CD40L antibodies. Cells were analyzed on a fluorescence activated cell sorter purchased from Becton Dickinson (San Jose, Calif.).

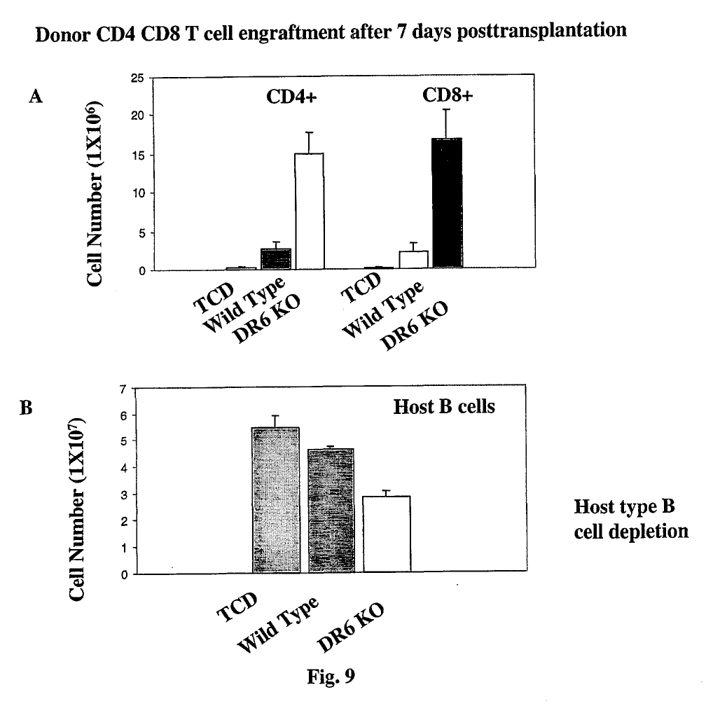

Splenocytes and thymocytes were analyzed with fluorescent conjugated antibodies against CD4, CD8, and CD19 at 7 days post-transplant to determine donor T cell expansion and host B cell depletion. Spleens from recipients of TCD grafts contained only small numbers of donor T cells as expected while recipients of wild type grafts contained at least 5 fold greater numbers of donor T cells (FIG. 9A). Donor T cell expansion was even further elevated in recipients of DR6 deficient grafts (FIG. 9A). Compared to wild type donors, DR6 deficient donors showed about a 5-fold increase in CD4⁺ cells and about a 25-fold increase in CD8⁺ cells (FIG. 9A). Host B cell numbers in the spleen were depressed at 7 days post-transplant for wild type donors compared to TCD donors as expected (FIG. 9B). Host B cell depletion was even greater for DR6 deficient donors (FIG. 9B). The percentage of cells with the T cell activation markers CD25, CD28, or CD40L was at least two-fold higher in mice receiving DR6 deficient grafts vs. wild type or TCD at 5 days post-transplant. The percentages of CD25 for TCD, wild type, and DR6 deficient were 7.4%, 7.5%, and 15%, respectively (data not shown). The percentages of CD28 for TCD, wild type, and DR6 deficient were 3.8%, 6.5% and 14%, respectively (data not shown). The percentages of CD40L for TCD, wild type, and DR6 deficient were 4.8%, 4.9%, and 12%, respectively (data not shown).

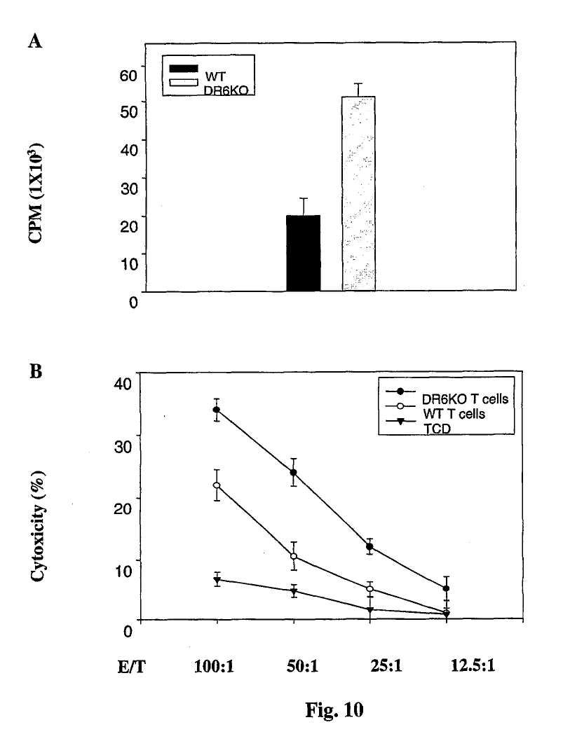

Splenocytes from Balb/c mice (H-2K^{d}) were irradiated with 3000 rads of a ¹³⁷Cs source at a rate of 250 rads/min and used as allogeneic stimulator cells. Responder splenocytes from either wild type B6 or DR6 deficient mice (H-2K^{b}) were mixed with the stimulator cells to initiate the reaction. Both cell populations were seeded at 4×10⁵ cells/well of a 96 well tray in 0.2 mls of IMDM supplemented with 10% FBS, 2 mM glutamine, 100 U/ml Penicillin, and 100 ug/ml streptomycin. After 72 hours of stimulation, wells were pulsed with 1 μCi of ³H-thymidine and allowed to incubate a further 6 hours before harvesting the cells and counting. Cytotoxic activity was measured directly (without in vitro restimulation) from splenocytes of transplanted mice. Purified splenocytes were obtained at 7 days post-transplant and seeded into wells of a 96 well U-bottom plate at different effector:target ratios. Target cells were P815 mastocytoma cells (H-2K^{d}) with EL-4 thymoma cells (H-2K^{d}) as a syngeneic control. Cytotoxicity assays were set up and carried out with a Cytotox 96 non-radioactive kit purchased from Promega Biotec (Madison, Wis.). In vitro responses of DR6 deficient T cells and wild-type T cells to alloantigens were measured in a MLR and CTL assay (FIG. 10). In the MLR, splenocytes from DR6 deficient or wild type mice (H-2K^{b}) were stimulated with irradiated splenocytes from Balb/c mice (H-2K^{d}) and then pulsed with tritiated thymidine at 72 hours to measure proliferation. Proliferative responses were greater than two fold higher for DR6 deficient T cells compared to wild type T cells (FIG. 10A). For the CTL assay, BDF1 mice were transplanted with either TCD cells, wild type, or DR6 deficient T cells. At 7 days post-transplant, splenocytes were obtained and examined for cytotoxic responses against P815 mastocytoma cells (H-2K^{d}). CTL activity, as measured by % cytotoxicity, was higher for DR6 deficient recipients compared to wild type recipients at each effector:target ratio (FIG. 10B).

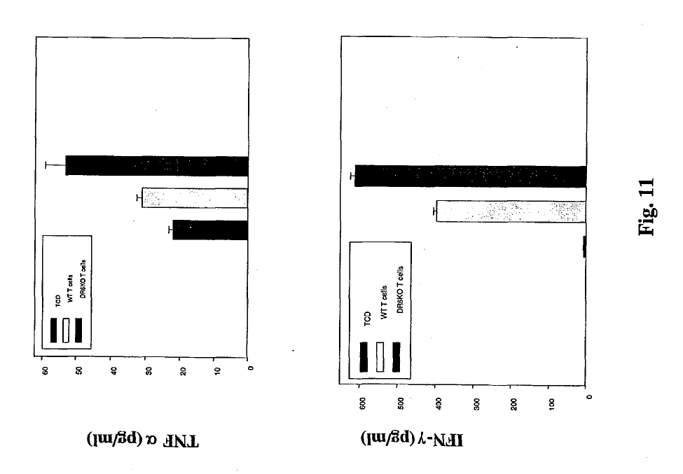

Serum was also obtained from mice at 7 days post-transplant and subjected to ELISA for TNF-α and IFN-γ. While recipients having wild type donors showed significant increases in serum TNF-α and IFN-γ compared to TCD recipient mice, recipients having DR6 deficient donors had significantly higher serum cytokine levels than recipients of T cells from wild-type mice. The average serum cytokine levels for TCD, wild type and DR6 deficient were 22, 31, and 54 pg/ml, respectively, for TNF-α and 10, 405, and 610 pg/ml, respectively, for IFN-γ (FIG. 11).

### EXAMPLE 28

### Treatment Protocol for Immunodeficiency

This example includes a description of a proposed treatment protocol for asymptomatic, HIV positive subjects designed to improve immunological functioning.

The subjects in the experimental treatment model consist of 12 asymptomatic HIV-positive males, ranging in age from 21 to 55, and with CD4 counts of 400 to 500. The subjects have no medical condition requiring prescription drugs. For the duration of the treatment study, the subjects abstain from the use of alcohol and drugs, including over- the-counter medications and health supplements.

To establish each subject's baseline, total CD4 and CD8 counts and CD4/CD8 ratios are taken three times, each at 30-day intervals. Treatment is started on the day that the third baseline counts are completed. For the next 90 days, the subjects receive subcutaneous injections of a sDR6 polypeptide (1 mg/m² body surface area) three times per week. Total CD4s, CD8s, and CD4/CD8 ratios are taken every 30 days until completion of the 90-day treatment regimen. At the end of the 90-day treatment period, the subjects are weaned off clonidine hydrochloride. Post-treatment CD4 and CD8 counts, as well as CD4/CD8 ratios are taken every 30 days for another 90 days. An analysis of variance is done to compare the pre-treatment, treatment, and post-treatment data on total CD4s, CD8s, and CD4/CD8 ratios. A p value of<0.05 is considered significant.

TABLE II
Immunofluorescent profiles of T cell sub-populations

| | Thymus | | Spleen | | Lymph node | |
|---|---|---|---|---|---|---|
| | WT(1.6 × 10⁸)^{a} | DR6 KO(1.4 × 10⁸) | WT(1.2 × 10⁸) | DR6 KO(1.3 × 10⁸) | WT(5.9 × 10⁷) | DR6 KO(6 × 10⁷) |
| CD4⁺ | 9.4 ± 0.3%^{b} | 16.0 ± 0.2% | 20 ± 0.3% | 22.2 ± 0.4% | 47.5 ± 0.4% | 48.8 ± 0.2% |
| CD8⁺ | 2.5 ± 0.3% | 1.9 ± 0.1% | 13.0 ± 1% | 16.0 ± 2% | 15.5 ± 3% | 16.0 ± 2% |
| CD4⁺ CD8⁺ | 83.7 ± 4% | 77.3 ± 5% | 0.62 ± 0.09% | 0.52 ± 0.07% | 4.9 ± 0.6% | 5.5 ± 0.4% |

^{a}Total cell numbers
^{b}Percentage of individual cell population
Data represent the mean ± SE of quadruplicate samples. There were four mice in each group.

We claim:

1. A method of treating or preventing a T cell mediated condition in a mammal that comprises administering to said mammal a therapeutically effective amount of a pharmaceutical composition comprising a DR6 agonist.

2. The method of claim 1 wherein said condition is selected from the group consisting of aberrant apoptosis, GVHD, rheumatoid arthritis, asthma, eczema, atopy, inflammatory bowel disease, vasculitis, psoriasis, insulin-dependent diabetes mellitus, pancreatis, psoriasis, cancer, multiple sclerosis, Hashimoto's thyroiditis, Graves disease, transplant rejection, systemic lupus erythematosus, autoimmune nephropathy, autoimmune hematopathy, idiopathic interstitial pneumonia, hypersensitivity pneumonitis, autoimmune dermatosis, autbimmune cardiopathy, autoimmune infertility, Behcet's disease, autoimmune gastritis, fibrosing lung disease, fulminant viral hepatitis B, fulminant viral hepatitis C, autoimmune hepatitis, chronic hepatitis, chronic cirrhosis, H. pylori-associated ulceration, organ rejection after transplantion, chronic glomeruonephritis, thrombotic thrombocytopenic purpura (TTP) and hemolytic uremic syndrome (HUS), aplastic anemia, myelodysplasia, multiple organ dysfunction syndrome (MODS), adult respiratory distress syndrome (ARDS), and at least one condition or symptom related thereto.

3. The method of claims 1-2 wherein the DR6 agonist is an agonistic anti-DR6 antibody.

4. The method of claims 1-2 wherein the DR6 agonist is a small molecule.

5. A method of treating or preventing a T cell mediated condition in a mammal that comprises the administering to said mammal a therapeutically effective amount of a pharmaceutical composition comprising a DR6 antagonist.

6. The method of claim 5 wherein said condition is selected from the group consisting of immunodeficiency, aberrant apoptosis, bacterial infection, viral infection, microbial infection, complications of infection, HIV, HIV-induced lymphoma, HIV-induced AIDS, fulminant viral hepatitis B, fulminant viral hepatitis C, chronic hepatitis, chronic cirrhosis, H. pylori-associated ulceration, cytoprotection during cancer treatment, recuperation from chemotherapy, recuperation from irradiation therapy, and at least one condition or symptom related thereto.

7. The method of claims 5-6 wherein the DR6 antagonist is a small molecule.

8. The method of claims 7-6 wherein the DR6 antagonist is an antagonistic anti-DR6 antibody.

9. The method of claim 8 wherein the antagonistic anti-DR6 antibody is an antagonistic anti-DR6 human antibody.

10. The method of claims 5-6 wherein the DR6 antagonist comprises a sDR6.

11. The method of claim 10 wherein the sDR6 comprises a polypeptide as shown from amino acid 42 through 350 of SEQ ID NO:2.

12. A method of treating or preventing a Th2 cell mediated condition that comprises administering to said mammal a therapeutically effective amount of a pharmaceutical composition comprising a DR6 agonist.

13. A method for enhancing Th2 cell mediated immunity in a mammal that comprises administering to said mammal a therapeutically effective amount of a pharmaceutical composition comprising at least one DR6 antagonist.

14. The method of claim 13 wherein the DR6 antagonist is an antagonistic anti-DR6 antibody.

15. The method of claim 14 wherein the antagonistic anti-DR6 antibody is an antagonistic anti-DR6 human antibody.

16. The method of claims 13 wherein the DR6 antagonist comprises a sDR6.

17. The method of claim 16 wherein the sDR6 comprises a polypeptide as shown from amino acid 42 through 350 of SEQ ID NO:2.

18. The method of claim 13 wherein the DR6 antagonist is a small molecule.

19. A method for inhibiting T cell mediated immunity in a mammal that comprises administering to said mammal a therapeutically effective amount of a pharmaceutical composition comprising at least one DR6 agonist.

20. The method of claim 19 wherein the DR6 agonist is an agonistic anti-DR6 antibody.

21. The method of claim 20 wherein the agonistic anti-DR6 antibody is an agonistic anti-DR6 human antibody.

22. The method of claims 19 wherein the DR6 agonist is a small molecule.

23. The method of claims 19-22 wherein the administration of the DR6 agonist is subsequent to the mammal having a bone marrow or solid organ transplantation.

24. Use of a DR6 agonist in the manufacture of a medicament for treating or preventing at least one symptom associated with aberrant apoptosis, GVHD, rheumatoid arthritis, asthma, eczema, atopy, inflammatory bowel disease, vasculitis, psoriasis, insulin-dependent diabetes mellitus, pancreatis, psoriasis, cancer, multiple sclerosis, Hashimoto's thyroiditis, Graves disease, transplant rejection, systemic lupus erythematosus, autoimmune nephropathy, autoimmune hematopathy, idiopathic interstitial pneumonia, hypersensitivity pneumonitis, autoimmune dermatosis, autoimmune cardiopathy, autoimmune infertility, Behcet's disease, autoimmune gastritis, fibrosing lung disease, fulminant viral hepatitis B, fulminant viral hepatitis C, autoimmune hepatitis, chronic hepatitis, chronic cirrhosis, H. pylori-associated ulceration, organ rejection after transplantion, chronic glomeruonephritis, thrombotic thrombocytopenic purpura (TTP) and hemolytic uremic syndrome (HUS), aplastic anemia, myelodysplasia, multiple organ dysfunction syndrome (MODS), adult respiratory distress syndrome (ARDS), and at least one condition or symptom related thereto in a mammal.

25. Use of a DR6 antagonist in the manufacture of a medicament for treating or preventing at least one symptom associated with immunodeficiency, aberrant apoptosis, bacterial infection, viral infection, microbial infection, complications of infection, HIV, HIV-induced lymphoma, HIV-induced AIDS, fulminant viral hepatitis B, fulminant viral hepatitis C, autoimmune hepatitis, chronic hepatitis, chronic cirrhosis, H. pylori-associated ulceration, cytoprotection during cancer treatment, recuperation from chemotherapy, recuperation from irradiation therapy, and at least one condition or symptom related thereto in a mammal.

26. The method of claims 5-11 wherein said DR6 antagonist is administered to said mammal in a T cell stimulating amount.

27. The method of claims 5-11 wherein said DR6 antagonist is administered to said patient in a CD4⁺ T cell stimulating amount.

28. The method of claims 5-11 wherein said DR6 antagonist is administered to said patient in a Th2 cytokine production stimulating amount.

29. The method of claims 1-4 wherein said DR6 agonist is administered to said mammal in a T cell inhibiting amount.

30. The method of claims 5-11 wherein said DR6 agonist is administered to said patient in a CD4⁺ T cell inhibiting amount.

31. The method of claims 5-11 wherein said DR6 agonist is administered to said patient in a Th2 cytokine production inhibiting amount.

32. The method of claims 1-2 wherein the DR6 agonist is a naturally occurring ligand agonist of DR6.
